(19) United States
(12) Patent Application Publication
(10) Pub. No.: US 2003/0206834 A1
(43) Pub. Date: Nov. 6, 2003

(54) Replaceable scent and multimedia storage medium for use with a playback apparatus having electrostatic scents release

(76) Inventors: DahShiarn Chiao, Albertson, NY (US); Chien-An Chang, Taipei (TW)

Correspondence Address: DahShiarn Chiao, 11 Wood Avenue, Albertson, NY, 11507, US

(21) Appl. No.: 10/436,608

(22) Filed: May 9, 2003

Related U.S. Application Data
(63) Continuation-in-part of application No. 10/043,928, filed on Jan. 9, 2002.
Application No. 10/043,928 is a continuation-in-part of application No. 09/713,983, filed on Nov. 16, 2000, now Pat. No. 6,602,475.

(51) Int. Cl. A61L 9/00; A61L 9/03

(52) U.S. Cl. 422/124; 422/4; 422/5; 428/905

(57) ABSTRACT

The present invention concerns a scent-bearing and multimedia disk or cartridge for use with separate multimedia playback and scent recovery systems. The scent-bearing and multimedia disk or cartridge comprises at least one scent release unit for effectively releasing scent, an associated scent storage medium for storing at least one scent, and an encapsulated multimedia storage medium for storing multimedia and scent release control information. The scent-bearing and multimedia disk or cartridge contains various scent release means for use by an associated scent recovery/release and multimedia playback system. The scent recovery/release and multimedia playback system is designed to be a portable, hand-held, self-contained, battery operated electrostatic multiple scents releasing, with a replaceable scent-bearing and multimedia disk or cartridge. The scent related content and recovery information is used by the multimedia playback and scent recovery/release system to coordinate scent release with or without multimedia playback. Additional embodiments of the present invention comprise electrostatic, ultrasonic nebulizing, and thermal scent release systems and methods for effectively and efficiently releasing scent stored in the scent-bearing and multimedia disk or cartridge. Further embodiments of the present invention comprise methods and systems for integrating the multimedia playback and scent recovery/release system with other applications.

### CROSS REFERENCE TO RELATED APPLICATION

This application is a continuation-in-part of a prior U.S. application with Ser. No. 10/043,928, filed on Jan. 9, 2002 and entitled “MULTIMEDIA AND SCENT STORAGE MEDIUM AND PLAYBACK APPARATUS HAVING ELECTROSTATIC SCENT RELEASE FOR USING SAME”, which is hereby incorporated by reference in its entirety as if filly restated herein.

### FIELD OF THE INVENTION

The present invention concerns scent-bearing and multimedia medium for use in conjunction with multimedia systems having scent-dispersing capability, and in particular to new scent-bearing cartridges for use with a hand-held or miniature multimedia playback system.

### BACKGROUND OF THE INVENTION

The present invention concerns scent storage and release systems that are used to create olfactory sensations in, e.g., aromatherapy. These systems can be used either separately or in combination with multimedia playback systems. When scent storage and release systems are used in combination with multimedia playback systems, an immersive multi-sensory experience can be created. Such multi-sensory experiences are attractive not only for entertainment purposes, but also for therapeutic purposes.

The prior art concerning such scent storage and release systems, particularly with reference to those systems intended for use with current and contemplated multimedia systems, are best by a number of limitations. In particular, little thought has been given to how a scent storage and release system can be integrated into a hand-held or miniature multimedia playback system.

This is true on two counts. First, users of such systems will have certain expectations about how such systems should operate due to their experience with such hand-held or miniature multimedia devices, e.g., mobile phone, personal digital assistant, portable computing devices, and wearable miniature multimedia devices.

The best of these systems give users a high degree of mobility in assessing multimedia experience through portable and miniature design features. With these systems the user can customize multimedia playback and interact with a remote signal source (e.g. internet, server, LAN, or wireless LAN) in a wireless fashion. This provides for a desirable customized and assessable multimedia experience.

In contrast, scent storage and release systems that may be used in combination with multimedia sources show no appreciation of this desirable feature. For example, U.S. Pat. No. 6,053,738 to Jr. Ivey describes methods for controlling the creation of olfactory stimuli. Although it suggests that its methods may be used in combination with a multimedia source, there is no description about how this can be accomplished. In particular, there is no description of how multiple scent storage units and scent release system can be integrated or manufactured in a compact fashion. Further, there is no description of how to coordinated the arrangement of multiple scent storage units with the multiple scent release system so that various scent can be retrieved for future use in repeating the multiple scent release operation.

A second problem concerns the physical integration of scent storage and release systems with known or contemplated hand-held or miniature multimedia playback systems. This concerns both scent recovery and release systems for use in recovering and releasing scents stored in the scent storage media, and the scent storage media itself. There is little or no appreciation of the need to design scent storage media so that they can be easily combined with multimedia storage media either in an integrated package, or in separate, but combinable, packages.

In particular, there are well-known current and contemplated multimedia storage formats. For example, there is the encapsulated Type II DVD RAM format in which an optical DVD storage media is encapsulated in a rugged housing. There are also the standard DVD and CD optical disk formats. There has been little or no effort made in seeking to integrate scent storage media with these well-known multimedia storage formats. Such an heretofore unknown combination would provide a user with a compact and easy-to-use multimedia and scent storage medium that would be capable in combination with a scent release and multimedia playback system of creating a complete multimedia experience.

There also is a lack of appreciation of the need to design scent release technologies that can be used in close proximity to sensitive electronic equipment. Scents often are volatile oil-like substances that unless properly handled can hinder the fault-free operation of a scent recovery and multimedia playback system.

Improved scent release technologies are therefore desired. Suitable hardware for applying appropriate scent release technologies, e.g., electrostatic scent release, have not been developed heretofore. These scent release technologies must be designed to operate in conjunction with current and contemplated micro-machinery technologies in order to take advantage of having technically efficient, cost effective, widespread consumer applicability and appeal. Presently, there are no known designs suitable for combining electrostatic scent release with micro-machinery technology.

Another problem concerns the efficient storage and release of scents, fragrances, flavors, aromas and therapeutic agents so that the minimum amount of these often expensive compounds can be used to maximum effect. One aspect of this problem concerns the efficient storage of scent between scent release operations so that the characteristic smell or scent of the substance is preserved. In addition, as these compounds often are volatile, they will be escape over time unless retained in an effective manner.

From the user's perspective, a number of limitations have been identified in current scent release technology intended for use with a hand-held or miniature device. In particular, current scent release systems make no provision for allowing a user to control the smell range by a user-specified scent release range control means. In the audio and video field, users have been given a high degree of control over personal space issue. Users do not have this level of control over scent release intended to apply with a hand-held or miniature device.

Thus, those familiar with the prior art desire several heretofore unknown and uncontemplated systems and methods to resolve the current limitations in the prior art. In particular, those skilled in the art desire a scent disk or cartridge design incorporating scent release units that can be easily combined with current and contemplated hand-held device or wireless applications. In particular, the most desirable scent cartridge design should be easily integrated into hand-held-device-like module. Such a scent cartridge and scent recovery/release system design could be easily integrated into the housing of the hand-held device, providing for a single, easy-to-use scent- and multimedia bearing medium that, in conjunction with a multimedia playback system, is capable of creating an immersive multimedia experience, including visual, auditory and olfactory elements.

Those skilled in the art also desire new scent release technologies that improve the performance of scent release systems intended for use in multimedia environments. Particularly desirable are systems that preserve the inherent scent characteristics of the fragrance, aroma or flavor to be released, and in no way cross-contaminate them as part of the scent release process. Also desired are scent release operations that heighten the impact of the scent or fragrance by proper neutralizing operation between each different scent releasing, so that a user experiences encounters a heightened, improved olfactory sensation. The desired scent release technology also would efficiently store the fragrance, aroma or flavor between release operations, thereby not only reducing the need to replace the scent disk or cartridge but also creating unintended residual smell due to the evaporation of scent.

In summary, those skilled in the art desire a scent disk or cartridge design that is as easy- to-use, inexpensive, rugged and effective as other powerful mobile-device and multimedia technologies.

### SUMMARY OF THE INVENTION

The limitations of the prior art are overcome in embodiments of the present invention. A first embodiment of the present invention comprises a scent-bearing disk or cartridge. The scent-bearing cartridge further comprises a scent storage region, a scent release region, a scent conducting tube region and a multimedia storage region. The scent storage region comprises a scent storage structure and scent release micro-pumping structure. The scent release region comprises a scent collecting chamber and various scent release apparatus. The multimedia storage region may take the form of an encapsulated and removable optical media. The scent-bearing disk or cartridge further comprises a machine-readable memory that may take the form of flash memory or other solid-state storage technologies for use in storing scent identification information for identifying the scent stored in the scent-bearing disk or cartridge.

A second embodiment of the present invention comprises a specific structure for a scent-bearing region. The specific structure comprises a physical configuration that can be easily attached to or integrated with a hand-held device like a wireless communication device or a personal assistance device. The scent cartridge has a rectangular solid housing configuration that shares the same width and depth as one side of the hand-held device. In one variant of the second embodiment, the scent cartridge housing and hand-held device have snap attachment points so they can be snapped together to form a single unit.

The scent disk or cartridge of the first embodiment comprises at least one scent storage unit and at least one scent release unit. In one variant of the first embodiment, the scent storage unit comprises a scent reservoir, a scent release micro-pumping unit and a scent conducting tube connecting the scent reservoir to the scent release unit. The scent release micro-pumping unit comprises a nozzle plate for pumping scent through scent conducting tube during scent release operations to improve the flow of scent from the scent storage region to the scent release unit. The nozzle plate comprises a three dimensional region enclosed on all sides except having a scent release opening facing scent release unit for releasing scent from the scent storage region. The scent conducting tube and the scent reservoir provide sufficient air pressure to equalize with the air pressure over the opening of the nozzle plate after the scent-release operations, and thereby prevents scent from escaping from the scent-bearing disk or cartridge. In another variant of the first embodiment, the scent release micro-pumping means further comprises piezoelectric, thermal bubble, micro-valve, or electrophoresis scent pumping apparatus for conducting scent through scent conducting tube during scent release operations to improve the flow of scent from the scent storage region to the scent release unit. In a further variant of the first embodiment, the nozzle plate and the scent storage region further comprises an integrated or detachable nozzle plate and scent storage reservoir unit. In one variant of the first embodiment, scent release unit comprises a scent collecting chamber and scent releasing means for collecting and then releasing scent into air. In another variant of the method of the first preferred embodiment, several scents in various form (e.g. liquid, gel, or solid) may be released simultaneously for mixing and releasing at the scent collecting chamber.

In one variant of the first embodiment, the scent release unit further comprises an electrostatic scent release means. The electrostatic scent release means comprises an electrostatic discharge needle, electrostatic grid and associated wiring. The electrostatic discharge needle is positioned within the scent release unit, and during scent release operations, ionizes scent contained within the unit. The ionized scent is attracted to the electrostatic grid, thereby improving evaporation of scent.

In another variant of the first embodiment, the scent release unit further comprises an ultrasonic nebulizing scent release means. The ultrasonic nebulizing scent release means comprises a piezoelectric plate and associated wiring. The piezoelectric plate is positioned within the scent release unit, and during scent release operations, vaporizes scent contained within the unit.

In a further variant of the first embodiment, the scent release unit further comprises a thermal scent release means. The thermal scent release means comprises a thermal heater plate and associated wiring. The thermal heater plate is positioned within the scent release unit, and during scent release operations, vaporizes scent contained within the unit.

A third embodiment of the present invention comprises methods for using a replaceable scent-bearing cartridge along with a hand-held device to be a portable, self-contained, battery operated multiple scents recovery/release and multimedia playback system. The method uses a separate scent recovery/release and multimedia playback system to release scent from the replaceable scent cartridge and to recover multimedia information from the hand-held device. The method comprises the following steps: receiving an input command from a user to initiate scent release through the communication port of the hand-held device; recovering digital scent release and multimedia playback control information corresponding to the user input command from a user; interpreting the identity, time, and duration of scent release reflected in the digital scent release and multimedia playback control information; and releasing the proper scents at the proper time and for the proper duration from the scent cartridge . In one variant of the third embodiment, an additional step in which scent to be released is ionized or vaporized occurs. In another variant of the method of the third preferred embodiment, several scents may be released simultaneously for similar or differing durations. In further variants, an additional step comprising a scent neutralizing operation is added to the method. During the scent neutralizing step, a scent neutralizing agent, for example, ozone is released. The step would follow the release of one scent and precede the release of another.

A fourth embodiment of the present invention comprises an separate multimedia playback and scent recovery apparatus for use in conjunction with the first, second and third embodiments, and in particular scent-bearing and multimedia cartridge where the multimedia storage region takes the form of an encapsulated optical or magnetic multimedia storage device like a DVD or Flash RAM. The separate multimedia playback and scent recovery system further comprises a remote multimedia input means for receiving remote multimedia input signals; a multimedia and scent recovery editing means for editing multimedia playback and scent recovery sequences, and a recording means for recording the multimedia and scent recovery sequence information to rewritable media like, e.g., rewritable optical storage devices or rewritable solid state storage devices.

A fifth preferred embodiment of the present invention comprises a scent-bearing cartridge having electrostatic scent release. The scent-bearing cartridge of the fifth embodiment is divided into two portions, a scent-bearing portion and a scent micro-pumping portion. In one variant of the fifth preferred embodiment, the scent-bearing portion of the disk is divided into separate scent-bearing regions, each for storing a separate scent. The separate scent-bearing regions are in the form of arcs and are arrayed about the outer portion of the disk. The scent-bearing regions are generally enclosed structures except for a first opening to allow a scent micro-pumping means to pumping scent through scent conducting tube during scent release operations to improve the flow of scent from the scent storage region to the scent release unit. In another variant of the fifth preferred embodiment, the scent-bearing portion of the cartridge is divided into separate scent-bearing regions, each for storing a separate scent. The separate scent-bearing regions are in the form of square and are arrayed about the inner portion of the cartridge. The scent-bearing regions are generally enclosed structures except for a first opening to allow a scent micro-pumping means to pumping scent through scent conducting tube during scent release operations to improve the flow of scent from the scent storage region to the scent release unit. In further variants, an additional step comprising a scent degrading operation is added to the method. During the scent-degrading step, ion and ozone are released to ionize or degrade the scent retention over the pores of the nozzle plate, thereby removing any retention or residual of previously-released scent. The step would follow the release of ozone and precede the release of another scent.

In one variant of the fifth embodiment, the high voltage source for creating corona discharge to ionize scent is either unipolar or bipolar. In another variant, scent is collecting in a groove-structure scent collecting chamber. The groove structure on the bottom surface of the scent collecting chamber to divide multiple scent onto each individual collecting zone, whereby the groove structure hinder one scent from trespassing onto another collecting zone while scent is releasing into the scent collecting chamber. The electrostatic scent release unit comprises an electrostatic discharge needle for ionizing scent, fragrance, aroma, therapeutic agent, or flavor released in the scent collecting chamber region, and electrostatic wiring for supplying electric potential to the electrostatic discharge needle. The individual scent bearing regions also have a scent release means for retaining the scent in the scent-storing regions until the initiation of scent release operations. In a further variant of the fifth embodiment, the electrostatic scent release unit comprises a corona discharge pin for creating corona discharge to ionize therapeutic agent and increasing the level of electrostatic charge on the particle of the therapeutic agent to assist in depositing the inhalabe therapeutic agent within the respiratory tract of the user. In further variants, an additional step comprising a scent releasing range control operation is added to the method. During the scent releasing range control step, a controlled amount of scent neutralizing agent, for example, ozone is released in an air flow faster than the previously-released scent flow to assist in the process of catching up the previously-released scent within a controlled range, thereby neutralizing or degrading the previously-released scent molecules in the air flow; and preventing the scent molecules from trespassing over the controlled range. The step would follow the release scent neutralizing or degrading agent in various angles by automatically rotation of the scent release unit to assist in neutralizing or degrading of the previously-released scent, dust, or other particles within the controlled range.

A sixth preferred embodiment of the present invention comprises a scent-bearing cartridge having ultrasonic nebulizing scent release. The scent-bearing cartridge of the sixth embodiment is divided into two portions, a scent-bearing portion and a scent micro-pumping portion. In one variant of the sixth preferred embodiment, the scent-bearing portion of the disk is divided into separate scent-bearing regions, each for storing a separate scent. The separate scent-bearing regions are in the form of arcs and are arrayed about the outer portion of the disk. The scent-bearing regions are generally enclosed structures except for a first opening to allow a scent micro-pumping means to pumping scent through scent conducting tube during scent release operations to improve the flow of scent from the scent storage region to the scent release unit. In another variant of the sixth preferred embodiment, the scent-bearing portion of the cartridge is divided into separate scent-bearing regions, each for storing a separate scent. The separate scent-bearing regions are in the form of square and are arrayed about the inner portion of the cartridge. The scent-bearing regions are generally enclosed structures except for a first opening to allow a scent micro-pumping means to pumping scent through scent conducting tube during scent release operations to improve the flow of scent from the scent storage region to the scent release unit.

In one variant of the sixth embodiment, a orifice mounted above the piezoelectric vibration plate of the scent collecting chamber to assist in vaporizing scent into air through the pores of the orifice, whereby, the orifice with multiple pore (size less than 80 micron) will be able to retain the scent liquid within the scent collecting chamber under normal air pressure without activation by the bottom piezoelectric vibration plate. In another variant, a corona discharge pin mounted above the piezoelectric plate for ionizing ultrasonic-vibrated scent molecules to assist in the scent release process. In further variant, scent deposited within a special constructed cup or container is vibrated indirectly through water or other non-erosive liquid by the ultrasonic nebulizing scent release apparatus. The ultrasonic nebulizing scent release unit comprises a piezoelectric plate for vaporizing scent, fragrance, aroma, therapeutic agent or flavor released in the scent collecting chamber region, and wiring for supplying electric potential to the piezoelectric plate.

A seventh preferred embodiment of the present invention comprises a scent-bearing cartridge having thermal scent release. The scent-bearing cartridge of the seventh embodiment is divided into two portions, a scent-bearing portion and a scent micro-pumping portion. In one variant of the seventh preferred embodiment, the scent-bearing portion of the disk is divided into separate scent-bearing regions, each for storing a separate scent. The separate scent-bearing regions are in the form of arcs and are arrayed about the outer portion of the disk. The scent-bearing regions are generally enclosed structures except for a first opening to allow a scent micro-pumping means to pumping scent through scent conducting tube during scent release operations to improve the flow of scent from the scent storage region to the scent release unit. In another variant of the seventh preferred embodiment, the scent-bearing portion of the cartridge is divided into separate scent-bearing regions, each for storing a separate scent. The separate scent-bearing regions are in the form of square and are arrayed about the inner portion of the cartridge. The scent-bearing regions are generally enclosed structures except for a first opening to allow a scent micro-pumping means to pumping scent through scent conducting tube during scent release operations to improve the flow of scent from the scent storage region to the scent release unit.

In one variant, a corona discharge pin mounted above the thermal heater plate for ionizing thermal-vaporized scent molecules to assist in the scent release process. The thermal scent release unit comprises a thermal heater plate for vaporizing scent, fragrance, aroma, therapeutic agent, or flavor released in the scent collecting chamber region, and wiring for supplying electric potential to the thermal heater plate.

A eighth preferred embodiment of the present invention comprises an electromagnetic wave inducible voltage or power generator. The electromagnetic wave inducible voltage or power generator comprises a receiver for receiving electromagnetic wave from a remote source, a inducible electrical currents generator, and a high voltage generating circuit transforming a low voltage into a high voltage for assisting in the scent release from electrostatic scent release apparatus. In one variant of the eighth preferred embodiment, an electromagnetic wave scent release apparatus creates high energy electromagnetic wave to ionize the scent molecules in the scent collecting chamber.

A ninth preferred embodiment of the present invention comprises methods for using a scent-bearing and multimedia disk or cartridge having scent releasing means. The method uses a separate scent recovery/release and multimedia playback system to release scent and recover multimedia information from the scent-bearing and multimedia cartridge. The method comprises the following steps: receiving an input command from a user to initiate scent release and multimedia playback; recovering digital scent release and multimedia playback control information corresponding to the user input command from the scent-bearing and multimedia portion of the cartridge; initiating multimedia recovery and playback corresponding to the multimedia segment selected by the user; interpreting the identity, time, and duration of scent release reflected in the digital scent release and multimedia playback control information; releasing the proper scents at the proper time and for the proper duration from the scent-bearing and multimedia cartridge using scent releasing means. In variants of the method of the ninth preferred embodiment, several scents may be released simultaneously for similar, or differing durations. In other variants, an additional step comprising a scent neutralizing operation is added to the method. During the scent neutralizing step, a scent neutralizing agent, for example, ozone would be released. The step would follow the release of one scent and precede the release of another. In further variants of the method of the ninth preferred embodiment, a energizing or refreshing scent may be released in correspond with a user-inputted preprogram time of the scent recovery/release system to assist in increasing alertness of the user.

In one variant of the ninth preferred embodiment, a security system integrated scent recovery/release system may release scent to assist in the alarm or alert operation for the user. In response to an electromagnetic wave, a vibration, a sound, an infrared detection, or other means of determining activation within the vicinity of the system, a sensor integrated scent recovery/release system may release scent. In another variant of the ninth preferred embodiment, a driver fatigue monitoring system integrated scent recovery/release system may release scent to assist in increasing the alertness or preventing drowsiness of the driver while the monitoring system has been triggered. In other variants, an additional step comprising a driver handlebar integrated control unit is added to the method. A driver may manually input a control signal to the scent recovery/release system for releasing scent in the car through the handlebar integrated control unit. In further variants, an additional step comprising a driver seatbelt integrated fatigue monitoring system is added to the method. A seatbelt integrated fatigue monitoring system may monitor the heartbeat, pulse, breath, and blood pressure of the driver in determine the driver's fatigue condition, and a control signal sent to the scent recovery/release system for releasing scent to the driver while the drossiness condition has been detected by the monitor.

A tenth preferred embodiment of the present invention comprises a scent-bearing and multimedia disk or cartridge for use with a separate multimedia playback and scent recovery system. The scent-bearing and multimedia disk or cartridge comprises a scent storage region and an encapsulated multimedia storage medium. The scent storage region comprises at least one scent storage unit and at least one scent release unit. In one variant of the tenth embodiment, the scent storage unit comprises an absorbent material containing scent that surrounds the scent release unit. The scent release unit comprises a scent collecting chamber and cover. The scent collecting chamber is a three-dimensional depression in the housing of the scent disk or cartridge that is open at the bottom and thereby connected to the scent storage unit, and open at the top to permit scent release operations. The cover encloses the scent release unit between scent-release operations, and thereby prevents scent from escaping from the scent- and multimedia bearing disk or cartridge.

In one variant of the tenth embodiment, the scent release unit further comprises an electrostatic scent release means. The electrostatic scent release means comprises an electrostatic discharge needle, electrostatic grid and associated wiring. The electrostatic discharge needle is positioned within the scent release unit, and during scent release operations, ionizes scent contained within the unit. The ionized scent is attracted to the electrostatic grid, thereby improving evaporation of scent. The individual scent bearing regions also have a scent release means for retaining the scent in the scent-storing regions until the initiation of scent release operations.

In another variant of the tenth embodiment, the scent release unit further comprises an ultrasonic nebulizing scent release means. The ultrasonic nebulizing scent release means comprises a piezoelectric plate and associated wiring. The piezoelectric plate is positioned within the scent release unit, and during scent release operations, vaporizes scent contained within the unit. The individual scent bearing regions also have a scent release means for retaining the scent in the scent-storing regions until the initiation of scent release operations.

In a further variant of the tenth embodiment, the scent release unit further comprises a thermal scent release means. The thermal scent release means comprises a thermal heater plate and associated wiring. The thermal heater plate is positioned within the scent release unit, and during scent release operations, vaporizes scent contained within the unit. The individual scent bearing regions also have a scent release means for retaining the scent in the scent-storing regions until the initiation of scent release operations.

In one variant of the tenth embodiment, the scent release means comprises single release valves. In this variant, the single release valves permit gas flow in only one direction, i.e., they permit released scent to escape, and prevent atmospheric gases from entering the scent-storing regions.

In another variant of the tenth embodiment, the scent-bearing region further comprises a specific structure. The specific structure comprises a physical configuration that can be easily attached to or integrated with an encapsulated optical multimedia storage device like a Type II DVD-RAM cassette. The scent cartridge has a rectangular solid housing configuration that shares the same width and depth as the Type II housing. In one variant of the second embodiment, the scent cartridge housing and Type II cassette are integrated as a single unit. In another variant the Type II cassette and scent cartridge housing have snap attachment points so they can be snapped together to form a single unit.

An eleventh embodiment of the present invention comprises a micro-pump and electrostatic scent release nosepiece for releasing scent directly to the nose and/or mouth of a user. The micro-pump and electrostatic scent release nosepiece comprises a nose-bridge-mounted control unit placed directly above the nose-bridge of the user for transmitting the control signal, and flexible-plastic-covered wirings to connect the scent storage and micro-pump release unit. The scent storage and micro-pump release unit comprises a nosepiece housing that comfortably fits over the nose of a user, at least one scent release storage units, at least one micro-pump unit and at least one scent release unit. At least one scent storage unit, at least one micro-pump unit, and at least one scent release unit is positioned adjacent to the nose, to supply scent, fragrances, therapeutic agents, or aromas to the user's nose. At least one scent storage unit, at least one micro-pump unit, and at least one scent release unit is positioned adjacent to the mouth to supply flavors to the user's mouth. Micro-pump units comprises at least a scent conducting tube connecting to at least one scent storage unit. Electrostatic scent release units comprise at least an electrostatic discharge needle and associated wiring for ionizing scent, fragrance, aroma, therapeutic agent, or flavor. Electrostatic scent release units included in variants of the eleventh embodiment further comprise electrostatic grids for attracting ionized scent molecules to improve efficiency of scent released.

A twelfth embodiment of the present invention comprises a multimedia playback and electrostatic scent release desktop diffuser for releasing scent directly to the user. The multimedia playback and electrostatic scent release desktop diffuser comprises a display screen mounted on the housing to display multimedia information, a housing for containing scent-bearing cartridge, speakers to play back auditory information, a user input pad and interface and an electrostatic scent release unit. In one variant of the twelfth embodiment, the scent release means further comprises a special design nozzle head for pumping scent through the activation of a solenoid during scent releasing operation to improve the flow of scent from the scent storage region to the scent release unit. In another variant of the twelfth embodiment, the scent-bearing cartridge means further comprises an independently replaceable and customizing scent storage region for refilling and rearranging at least one scent by the user. Customizing the scent storage region means the scents can be related to each other in a manner which is not dependent on some other media, and/or are not designed to be used simultaneously with other media.

A thirteenth embodiment of the present invention comprises a multimedia playback and electrostatic scent release desktop diffuser for releasing scent directly to the user. The multimedia playback and electrostatic scent release desktop diffuser comprises a display screen mounted on the housing to display multimedia information, a separate multimedia playback and scent recovery system, a housing for containing scent-bearing cartridge, speakers to play back auditory information, a user input means and an electrostatic scent release unit. In one variant of the thirteenth embodiment, the separate multimedia playback and scent recovery system further comprises scent related content and scent release control information for the scent-bearing cartridge is provided by a separated multimedia storage medium from the multimedia playback system. In another variant of the thirteenth embodiment, the separate multimedia playback and scent recovery system further comprises scent related content and scent release control information for the scent-bearing cartridge is provided by a remote source (e.g. internet, server, LAN, or wireless LAN). In a further variant of the thirteenth embodiment, the scent-bearing cartridge further comprises a graphical or text labeling on the storage case to provide user with the scent related content, texts or recovery information. User-inputting scent related content and texts information the scents can be related to each other in a manner which is not dependent on some other media, and/or are not designed to be used simultaneously with other media.

A fourteenth embodiment of the present invention comprises a multimedia playback and electrostatic scent release desktop diffuser for releasing scent directly to the user. The multimedia playback and electrostatic scent release desktop diffuser comprises a docking structure for holding a hand-held device, a communication and power supply port to connect between the multimedia playback and electrostatic scent release desktop diffuser and the hand-held device, a housing for containing scent-bearing cartridge, speakers to play back auditory information, a user input pad and interface and an electrostatic scent release unit. In one variant of the fourteenth embodiment, the hand-held device further comprises a receiver for receiving wireless signal from a remote source and a transceiver to transmit the control signal to the multimedia playback and scent recovery system through the communication and power supply port.

A fifteenth embodiment of the present invention comprises a multimedia playback and electrostatic scent release personal diffuser for releasing scent directly to the user. The multimedia playback and electrostatic scent release personal diffuser comprises an attachment means placed directly adjacent to the housing of the diffuser, a display screen mounted on the housing to display multimedia information, an earphone port to output auditory information, a housing for containing scent-bearing cartridge, speakers to play back auditory information, a user input pad and interface and an electrostatic scent release unit. The attachment means comprises a clip structure, a drawstring, an interlocking, a loop of fabric, a VELCRO-like fabric patches, or a mechanical snap. Electrostatic scent release units comprise at least an electrostatic discharge needle and associated wiring for ionizing scent, fragrance, aroma, therapeutic agent, or flavor. A fan and ducting to create an air flow in the vicinity of the electrostatic discharge needle to assist in entraining scent ionized by the electrostatic discharge needle. Electrostatic scent release units included in variants of the fifteenth embodiment further comprise electrostatic grids for attracting ionized scent molecules to improve efficiency of scent released.

One of ordinary skill in the art will understand that each of the alternate embodiments can be practiced either singly, in combination, or in combination with other scent release and multimedia playback systems. In addition, one of ordinary skill in the art will understand that the preceding alternate embodiments can be combined in various ways to accomplish different system objectives. Further, one of ordinary skill in the art will understand that the means of accomplishing the functionality described herein can be distributed in various ways, e.g., combined into a single stand-alone system or distributed among a plurality of separate, but interconnected and communicating, systems. All of these variants are within the scope of the present invention.

Thus it is seen that the multimedia and scent storage cartridge design having electrostatic scent release overcomes the limitations of the prior art. For example, the scent storage design provides a rugged and easily-integrated unit that can be used in combination with Type II DVD RAM cartridges or hand-held devices. The scent cartridge design takes advantage of an existing optical storage format and hand-held device accessory module design and provides a scent release capability. The scent cartridge design will also permit designers to add scent release capability to existing multimedia playback systems.

The present invention also provides an electrostatic scent release system for use in combination with the scent cartridge. The electrostatic scent release system of the present invention provides for efficient and effective scent release that creates a pleasing olfactory sensation for users with a minimum of expensive fragrance, scent, therapeutic agent, aroma and/or flavor.

### BRIEF DESCRIPTION OF THE FIGURES

The above and other objects and advantages of this invention will be apparent upon consideration of the following detailed description, taken in conjunction with the accompanying drawings in which like characters refer to like elements throughout and in which:

FIG. 1 depicts side, top and perspective views of a scent enable hand-held device and scent-bearing cartridge made in accordance with a preferred embodiment of the present invention;

FIG. 2 depicts side, top and exploded views of a scent enable hand-held device and scent-bearing cartridge made in accordance with a preferred embodiment of the present invention;

FIG. 3 depicts perspective views of alternate scent enable hand-held device and scent-bearing cartridge configurations made in accordance with a preferred embodiment of the present invention;

FIG. 4A depicts the construction of the scent-bearing cartridge of a preferred embodiment of the present invention;

FIG. 4B depicts alternate scent-bearing cartridge made in accordance with a preferred embodiment of the present invention;

FIG. 4C depicts alternate scent-bearing cartridge made in accordance with a preferred embodiment of the present invention;

FIGS. 5A-5D depict in cross-sectional view of a scent collecting chamber having an electrostatic plate, a piezoelectric plate, or a thermal plate made in accordance with a preferred embodiment of the present invention;

FIGS. 6A-6H depict in cross-sectional view of a micro-pumping unit constructed in accordance with a preferred embodiment of the present invention;

FIG. 7 depicts in cross-sectional views of alternate scent-bearing cartridge release units constructed in accordance with a preferred embodiment of the present invention;

FIG. 8 depicts in cross-sectional view of a piezoelectric scent release unit constructed in accordance with a preferred embodiment of the present invention;

FIG. 9 comprises a block diagram depicting the operation of a electromagnetic wave induced high voltage generation operating in accordance with the method of a preferred embodiment of the present invention;

FIGS. 10 and 11 depict in schematic form the operation of scent releasing range control operating in accordance with a preferred embodiment of the present invention;

FIG. 12 depicts in schematic form the operation of automatically rotation scent neutralization and degradation operating in accordance with a preferred embodiment of the present invention;

FIGS. 13 and 14 comprise a block diagram depicting the operation of scent releasing range control operating in accordance with the method of a preferred embodiment of the present invention;

FIG. 15 comprises a block diagram depicting the operation of security system integrated scent recovery/release system operating in accordance with the method of a preferred embodiment of the present invention;

FIG. 16 comprises a block diagram depicting the operation of driver fatigue monitoring system integrated scent recovery/release system operating in accordance with the method of a preferred embodiment of the present invention;

FIG. 17 depicts in schematic form the operation of driver handlebar integrated control unit and the scent recovery/release system in accordance with a preferred embodiment of the present invention;

FIG. 18 depicts in schematic form the operation of driver seatbelt integrated fatigue monitoring system and the scent recovery/release system in accordance with a preferred embodiment of the present invention;

FIG. 19 comprises a block diagram depicting the operation of sensor integrated scent recovery/release system operating in accordance with the method of a preferred embodiment of the present invention;

FIGS. 20A-20C depict in cross-sectional views various scent release means constructed in accordance with a preferred embodiment of the present invention;

FIGS. 21A and 21B depict top and perspective views of a scent-bearing and multimedia disk having various scent release means constructed in accordance with a preferred embodiment of the present invention;

FIGS. 22A and 22B depict top and perspective views of an alternate scent-bearing and multimedia disk having various scent release means made in accordance with a preferred embodiment of the present invention;

FIG. 23 depicts a cross-sectional view of an alternate scent-bearing cartridge having various scent release means made in accordance with a preferred embodiment of the present invention;

FIG. 24 depicts a see-through perspective view of an alternate scent-bearing cartridge having various scent release means made in accordance with a preferred embodiment of the present invention;

FIG. 25 depicts an alternate scent-bearing and multimedia disk made in accordance with the a preferred embodiment of the present invention;

FIG. 26 depicts side, top and perspective views of a Type II DVD cassette compatible scent-bearing cartridge made in accordance with a preferred embodiment of the present invention;

FIG. 27 depicts side, top and perspective views of a scent-bearing cartridge with a data storage medium (e.g., SIM card, flash memory, memory chip) and a scent release unit made in accordance with a preferred embodiment of the present invention;

FIG. 28A depicts a micro-pump and electrostatic scent release nosepiece made in accordance with a preferred embodiment of the present invention;

FIG. 28B depicts an alternate micro-pump and electrostatic scent release nosepiece made in accordance with a preferred embodiment of the present invention;

FIG. 29 depicts side, top and perspective views of the multimedia playback and electrostatic scent release desktop diffuser made in accordance with a preferred embodiment of the present invention;

FIG. 30 depicts an alternate scent storage region and the multimedia playback and electrostatic scent release desktop diffuser made in accordance with a preferred embodiment of the present invention;

FIG. 31 comprises a block diagram depicting the operation of the multimedia playback and electrostatic scent release desktop diffuser operating in accordance with the method of a preferred embodiment of the present invention;

FIG. 32 comprises a block diagram depicting the operation of a graphical or text labeled scent storage case operating in accordance with the method of a preferred embodiment of the present invention;

FIG. 33 comprises a block diagram depicting the operation of a graphical or text labeled scent'storage case operating in accordance with the method of a preferred embodiment of the present invention;

FIGS. 34A-34C depict in cross-sectional view of various special design nozzle heads constructed in accordance with a preferred embodiment of the present invention;

FIG. 35A depicts perspective views of a hand-held device and the alternate multimedia playback and electrostatic scent release desktop diffuser made in accordance with a preferred embodiment of the present invention;

FIG. 35B depicts side, top and perspective views of a hand-held device and the alternate multimedia playback and electrostatic scent release desktop diffuser made in accordance with a preferred embodiment of the present invention; and

FIG. 36 depicts perspective views of a multimedia playback and electrostatic scent release personal diffuser made in accordance with a preferred embodiment of the present invention.

### DETAILED DESCRIPTION OF THE PREFERRED EMBODIMENTS

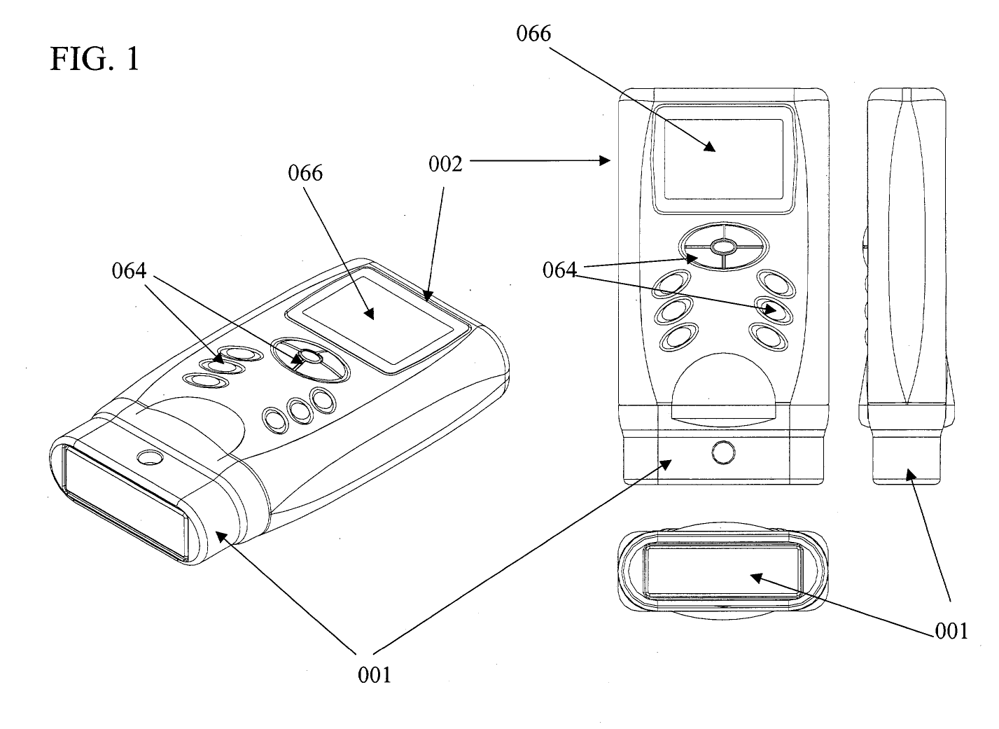

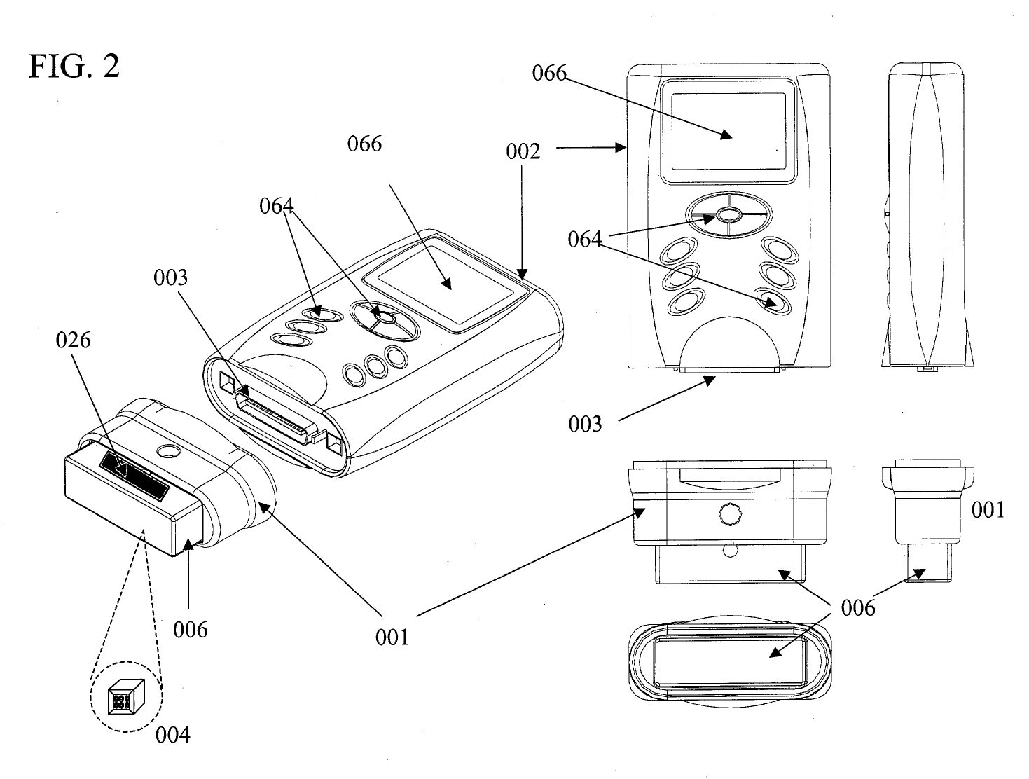

A scent-bearing disk or cartridge made in accordance with the second and third preferred embodiments of the present invention is depicted in perspective, top and side views in FIG. 1. The hand-held device integrated scent-bearing cartridge 001 is depicted attached to a hand-held device 002. The hand-held device is shown to better advantage in FIG. 2, which depicts the communication and power supply port 003 of the hand-held device detached so the hand-held device integrated scent-bearing disk or cartridge can be removed or reinserted.

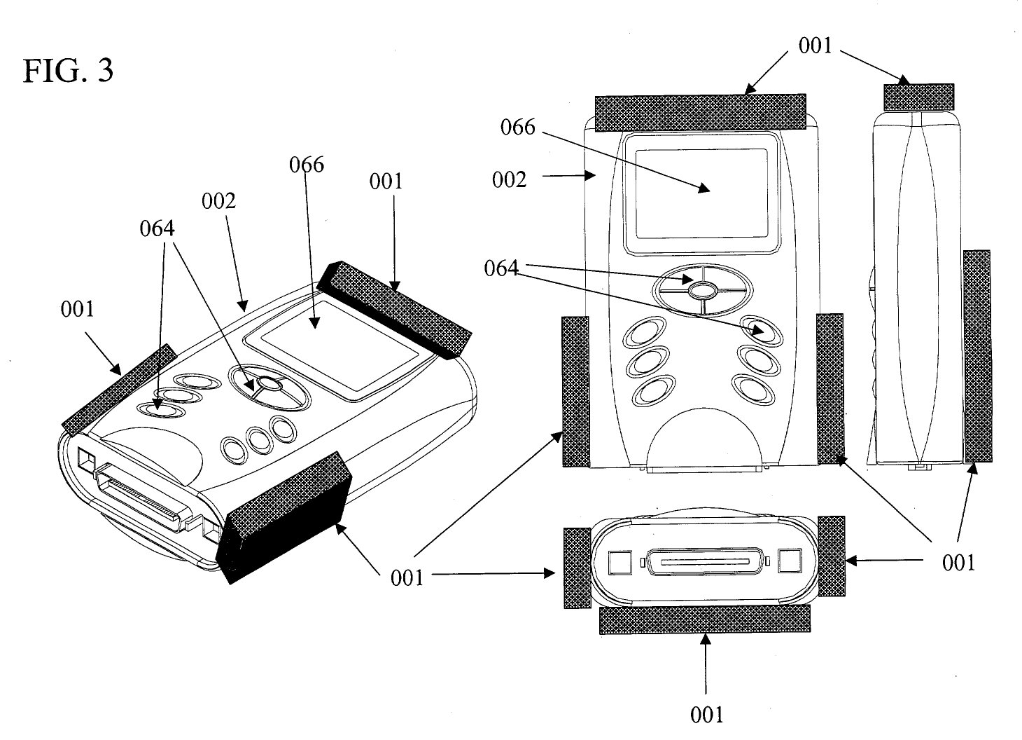

FIGS. 3 depicts variants of the first preferred embodiment in which the hand-held device integrated scent-bearing cartridge 001 are combined in various ways about the periphery of the hand-held device 002.

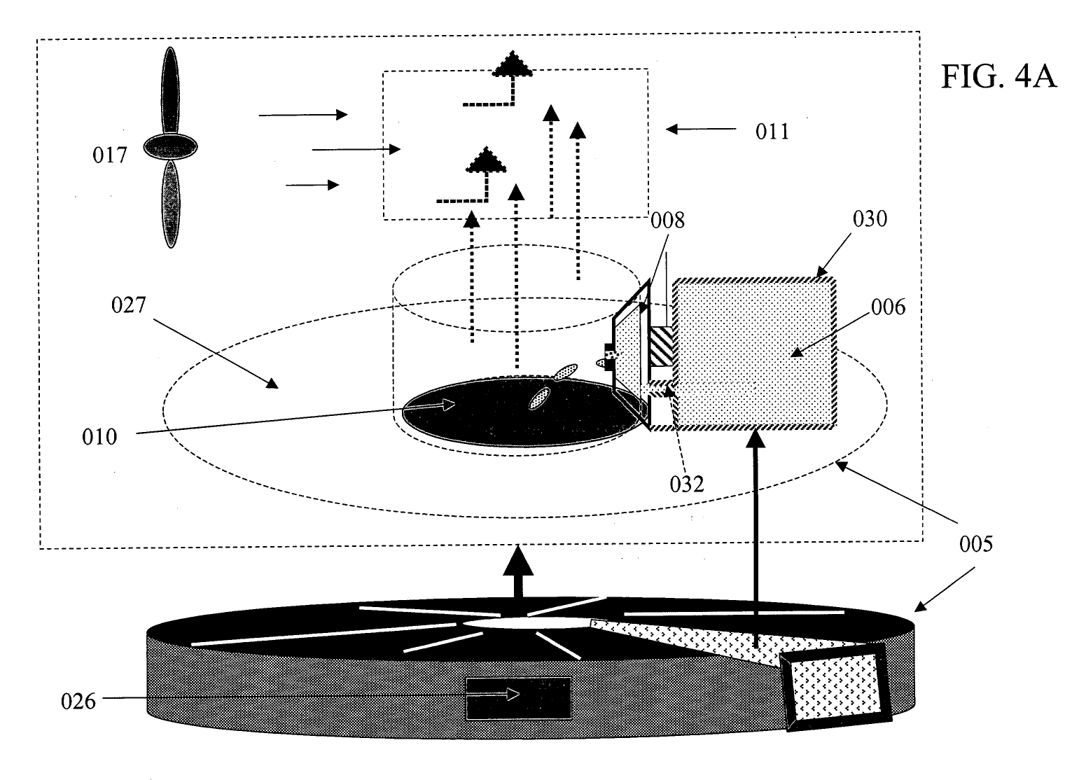

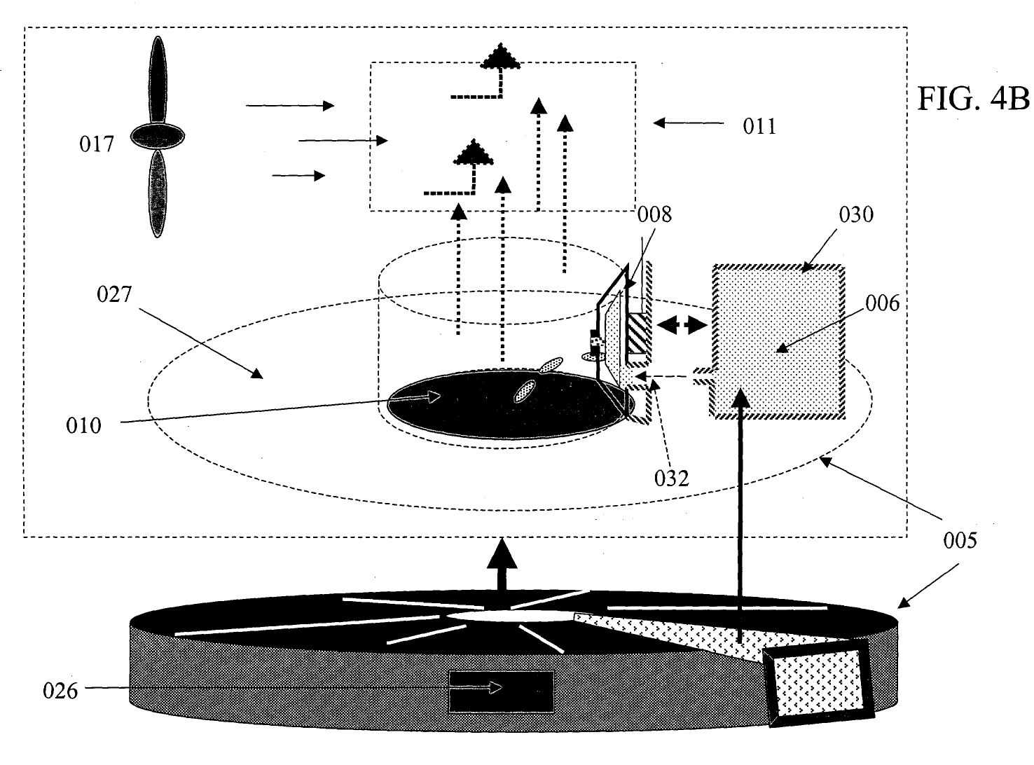

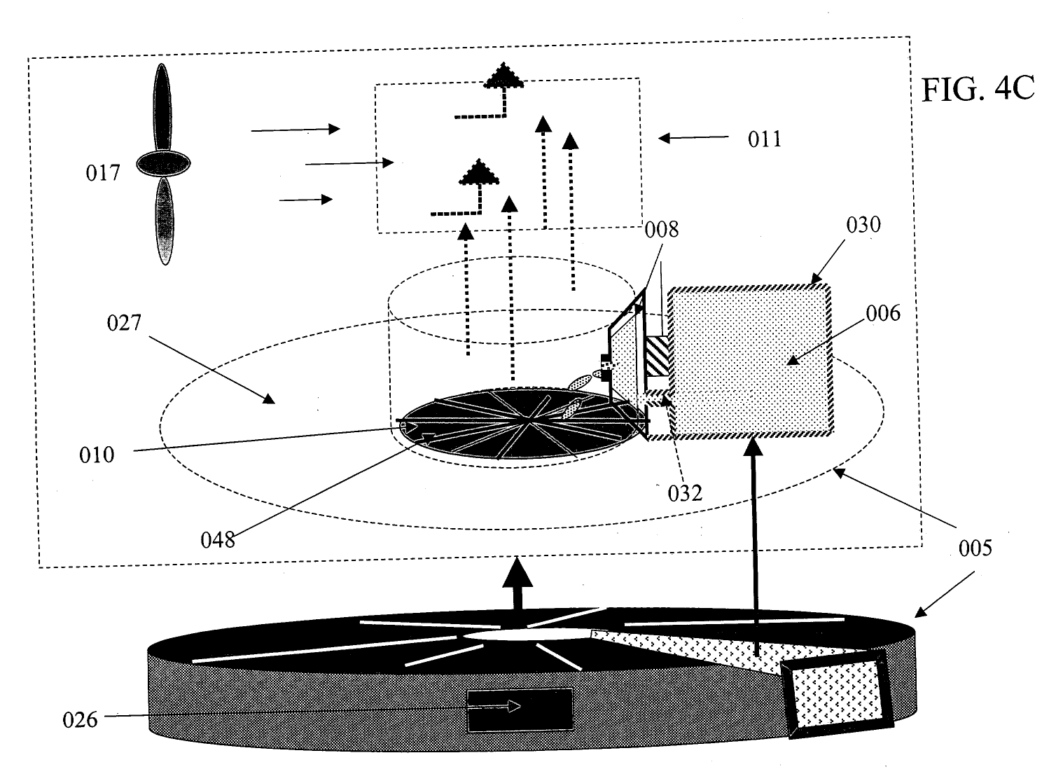

A complete scent-bearing cartridge 005 having scent release micro-pumping units made in accordance with the first preferred embodiment of the present invention is shown in FIG. 4A. The scent-bearing cartridge 005 shown in FIG. 4A comprises a plurality of separate scent release micro-pumping units 030, each of which is contained in a three-dimensional sector of the disk. The scent release micro-pumping units 030 has a scent conducting tube 032 that is continuously in directly connect with the scent storage reservoir 006. Delivery of an electric charge through the nozzle plate 008 sublimates the scent into many scent or drug droplets which laid on the scent collecting chamber 010. After a sufficient quantity of the scent droplets has been collected, the measured quantity of scent droplets is ionized by unipolar or bipolar electrostatic charge into the scent venting chamber 011. As above, once the ionized scent vapor has been delivered into the venting chamber 011, a signal causes the fan or air pump 017 to blow in a series of air flows, each of which can force a ionized scent vapor of either neutralized by a grid or non-neutralized to be expelled through the opening at the end of the venting chamber 011. The scent vapors are directed at the general vicinity of the user's face, and more particularly at the user's nose.

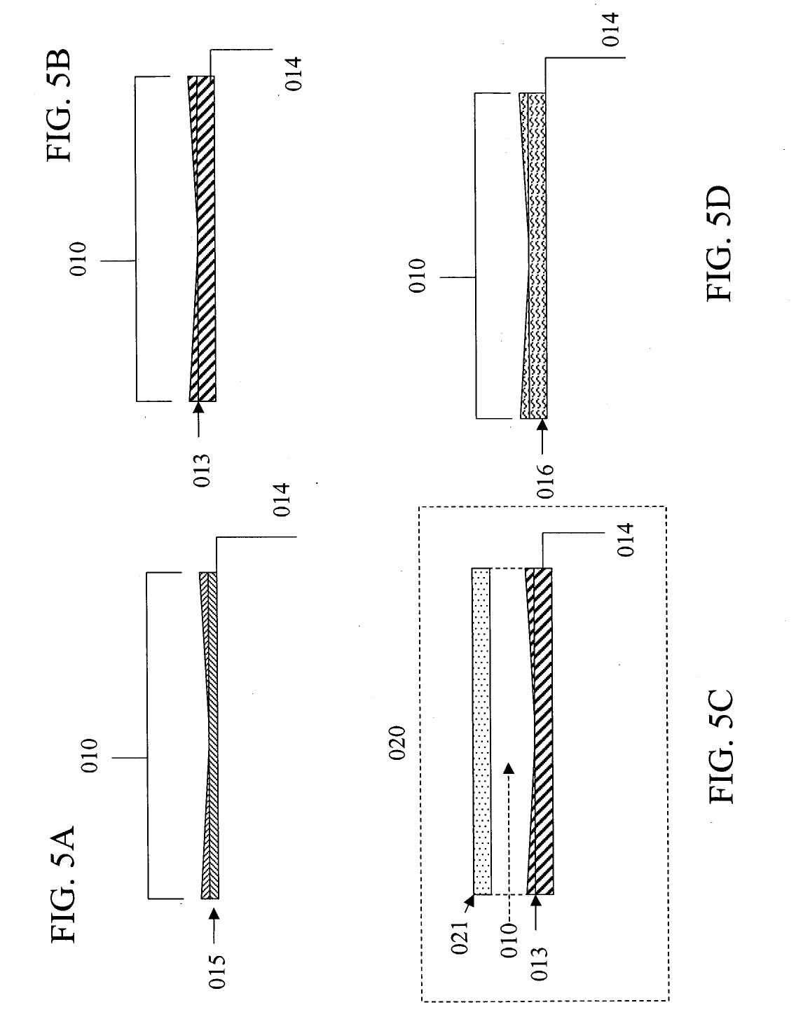

In one alternative embodiment shown in FIG. 5A,an electrostatic apparatuses having wiring 014 can be integrated into the scents collecting chamber 010 to ionize single or multiple scent liquid into scent molecules. Each individual droplet can be applied through a micro-pumping means, a wicking method, an elastomer funnel, or by some other droplet placing means. Formation of an ionized scent creates very small, precisely measured droplets of the scent which optimizes the surface area relative to the volume. Optimization of the size of the scent improves the vaporization rate and the dispersion rate of the scent into the air.

In another alternative embodiment shown in FIGS SB and 5C, a piezo-ceramic vibration plate 013 or a piezoelectric scent pumping apparatus 020 having a pored-orifice 021 or without having a pored-orifice 21can be integrated into the scents collecting chamber 010 to sublimate single or multiple scent liquid into a vapor. Each individual droplet can be applied through a micro-pumping means, a wicking method, an elastomer funnel, or by some other droplet placing means.

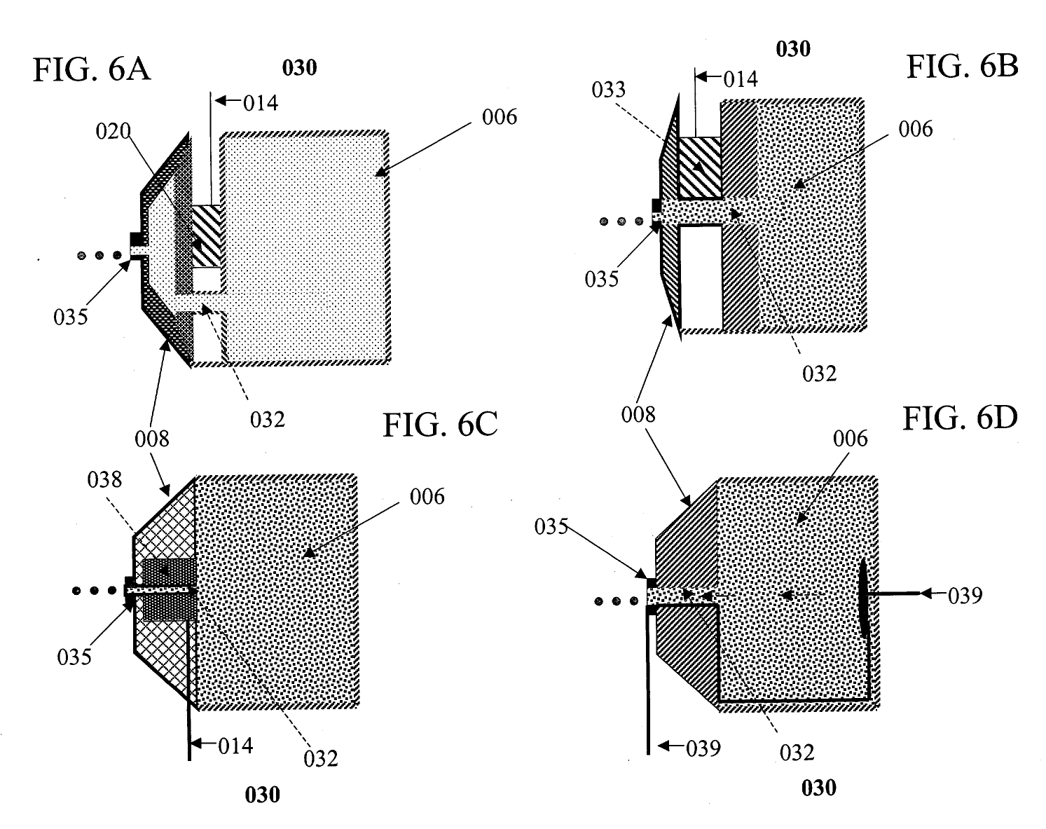

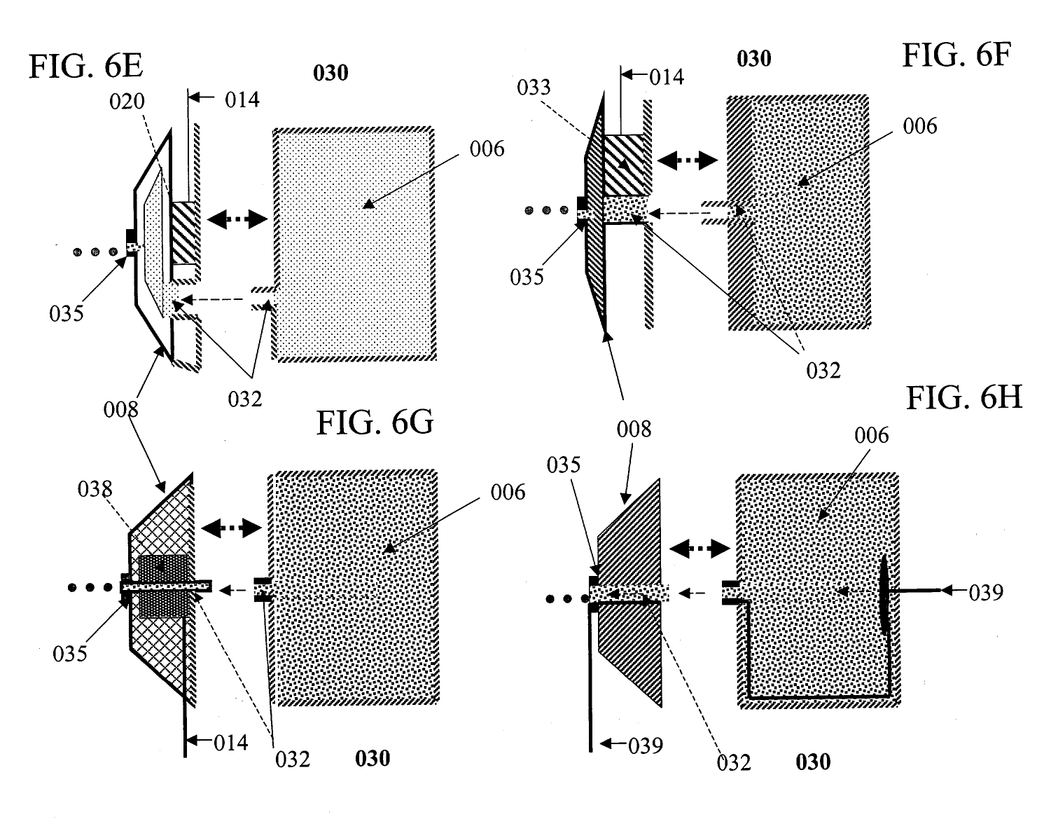

In a further alternative embodiment shown in FIG. 5D, a thermal plate 016 having wiring 014 can be integrated into the scents collecting chamber 010 to sublimate single or multiple scent liquid into a vapor. Each individual droplet can be applied through a micro-pumping means, a wicking method, an elastomer funnel, or by some other droplet placing means. In yet other embodiment of the present invention, the scent release micro-pumping unit 030 can also include at least one of a piezoelectric scent pumping 020, as showed in FIG. 6A, a thermal bubble scent pumping 033, as showed in FIG. 6B, a micro-valve scent pumping 038, as showed in FIG. 6C, or an electrophoresis scent pumping 039, as showed in FIG. 6D, to deliver a vapor or droplet scent chemical to the scent collecting chamber 010 through the scent conducting tube 032. A small amount of the scent chemical can be expelled in a droplet form to the scent collecting chamber 010 through a opening or orifice 035 from a scent release micro-pumping unit. The scent release micro-pumping unit 030 produces droplets that are typically in the range of 10 microns to 20 microns. The droplets are moved into the scent collecting chamber 010 where the droplets are ionized or vaporized and then sprayed into the venting chamber 011.

In a preferred embodiment of the present invention, top substrate of the nozzle plate 008 is formed of silicon as it may be micro-machined to obtain a very high precision manufacturing, such a high precision and absence of leachable components being much more difficult to obtain with plastics or the like, for instance by using UV exposure or a plasma etching treatment of various plastic material. The bottom substrate of the nozzle plate 008 is etched so as to form a thinner middle section which may act as a membrane for transmitting a vibration or heat. Bottom substrate may be made of glass, ceramics, silicon, high-density polymer or the like. Top substrate of the nozzle plate 008 preferably consists of silicon, but it may consist of plastic, high density polymer, ceramics, metal or silicon or the like for its main body. As mentioned, outlet pore 035 is formed in top substrate, so that the excited scent 007 may leave the scent release micro-pumping unit 030 as a droplet spray. To this effect, this top substrate is micro-machined, for example in a well known anisotropic etching manner at several places, by using an appropriate mask. By using the usual micro-machining techniques, anisotropic etching is used, thus resulting in straight or cylindrical or of another suitable geometrical shape.

It is another advantage of using the scent collecting chamber 010 for mixing multiple scents in either gaseous phase or liquid phase. Multiple scents can be delivered into the scent collecting chamber at various quantity through each micro-pumping means to achieve the liquid phase mixing before the vaporization been start by a electrostatic, a ultrasonic nebulizing or a thermal scent release apparatus. To having gaseous phase scent mixing, multiple scents can be delivered into the activated scent release unit at various quantities through each micro-pumping means.

In order to let ionized scent or therapeutic agent to stick onto the internal surface of nose, it is known that the site of deposition within the respiratory tract of an inhalable substance can be influenced by the level of electrostatic charge on the particles of the inhalable substance. In addition, after electrically charged, they will rapidly deposit on the lining of the nasal cavity or within the upper respiratory tract thereby ensuring that penetration of the particles is limited.

This is particularly important in the case of for example therapeutic agents to be administered to the upper respiratory tract (e.g. the nasal cavity) because such therapeutic agents may be unsuitable or induce an adverse reaction if inhaled into the lungs. With other materials however it is desirable to deliver the material in a controlled manner to ensure application to the specific area to be treated and/or to improve the efficiency at which the active ingredients are delivered by minimizing the amount of spray outside the target.

It is yet another advantage of using electrostatic to release scent. The ion can clean off the scent retention over the pore of nozzle plate, so that there will not be any retention or residual of scents after each scent releasing.

Another object of the present invention is to reduce manufacturing problems and/or costs by providing a disposable scent-bearing and multimedia disk or cartridge in which the nozzle plate, scent conducting tube and scent storage region can be manufactured as one integral part or separate parts.

In an alternative scent-bearing cartridge 005 embodiment shown in FIGS. 4B, 6E, 6F, 6G, and 6H, the scent storage reservoir 006 can be detached from the micro-pumping unit 030 and replaced with other compatible scent storage reservoir 006 by a user.

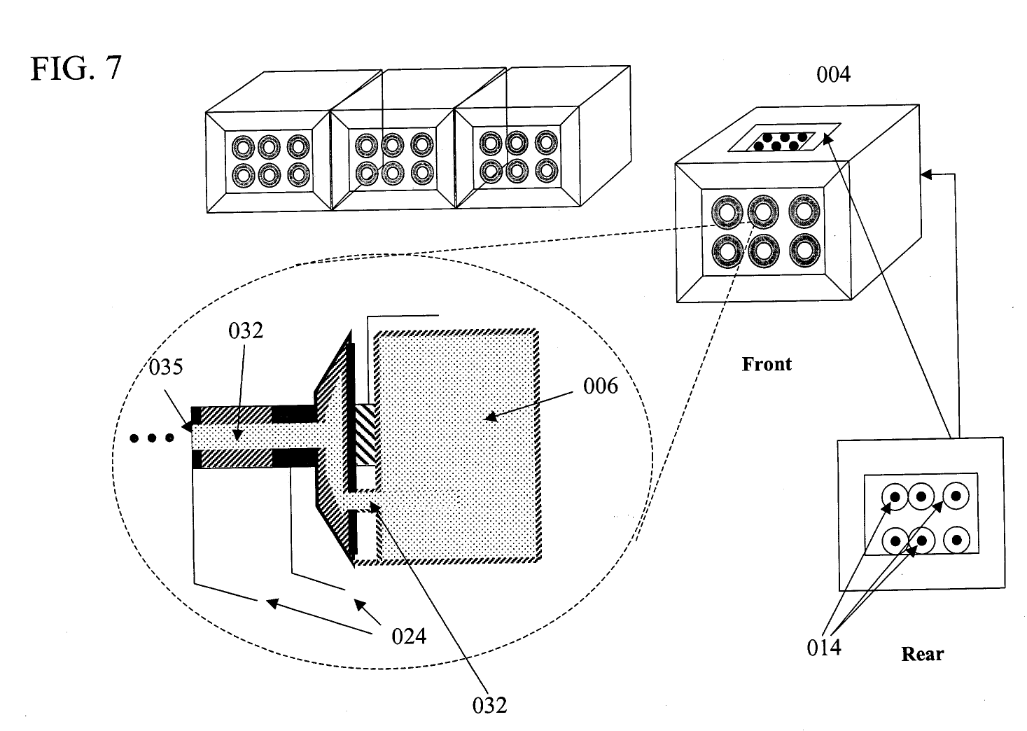

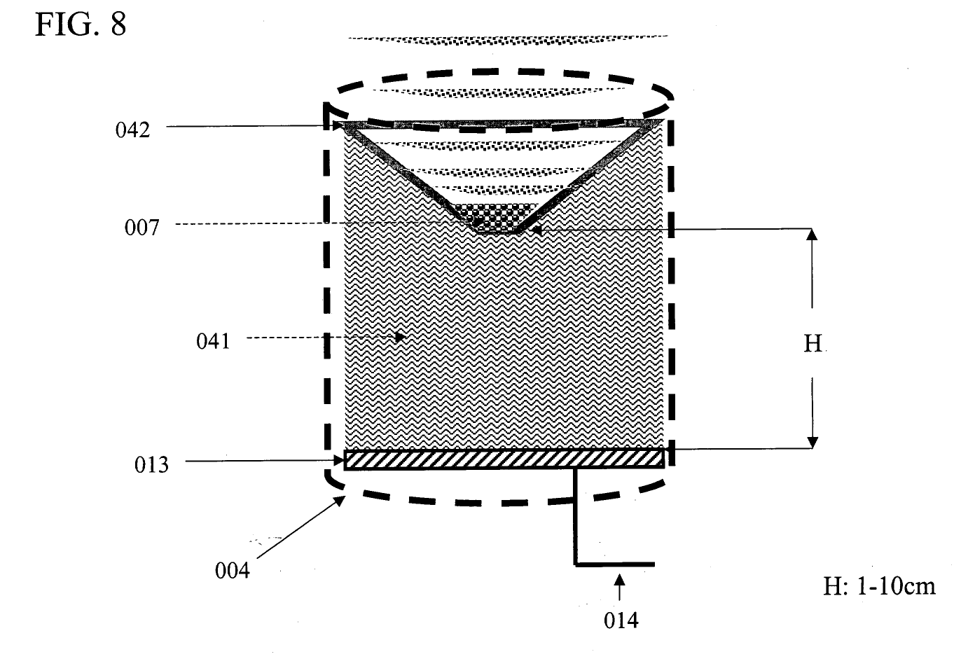

A complete scent release unit 004 having at least one scent release micro-pumping unit 030 made in accordance with the first preferred embodiment of the present invention is shown in FIG. 7. The scent conducting tube 032 can also be formed at the front of the scent release micro-pumping unit to thereby focus the charge from the electrostatic electrode 024 through this scent conducting tube 032 and to the product prior to its dispersal to nozzle tip 035. Scent is drawn from the scent storage region 006 across the micro-pumping unit 030 towards the outer peripheral surface 035 of the tube. Because of the intensified electric field 024 prevailing in the vicinity of the leading extremity, the liquid is preferentially drawn into one or more ligaments from an edge portion in the locality of extremity and thereafter breaks up within the scent storage and micro-pump release unit 053 into droplets to form the spray. In addition, the asymmetry wiring arrangement can provide multiple scent release units 004 in assembly for a hand-held device integrated scent-bearing cartridge 001, scent bearing and multimedia disk 012, micro-pump and electrostatic scent release nosepiece 050, or many other applications. In an alternative embodiment shown in FIG. 8, a piezoelectric vibration plate 013 can be integrated into the bottom of a scent release unit 004 to sublimate scent liquid 007 in anti-erosive plastic cup or container 042 (e.g. PET, PP etc.) into scent vapor through the transmitting material (e.g. water) 041. The ultrasonic vibration can be transmitted into the anti-erosive plastic cup 042 through water or other non-erosive liquid 041 by a certain distance (e.g. 110 cm) varied by different vibration frequency or angle.

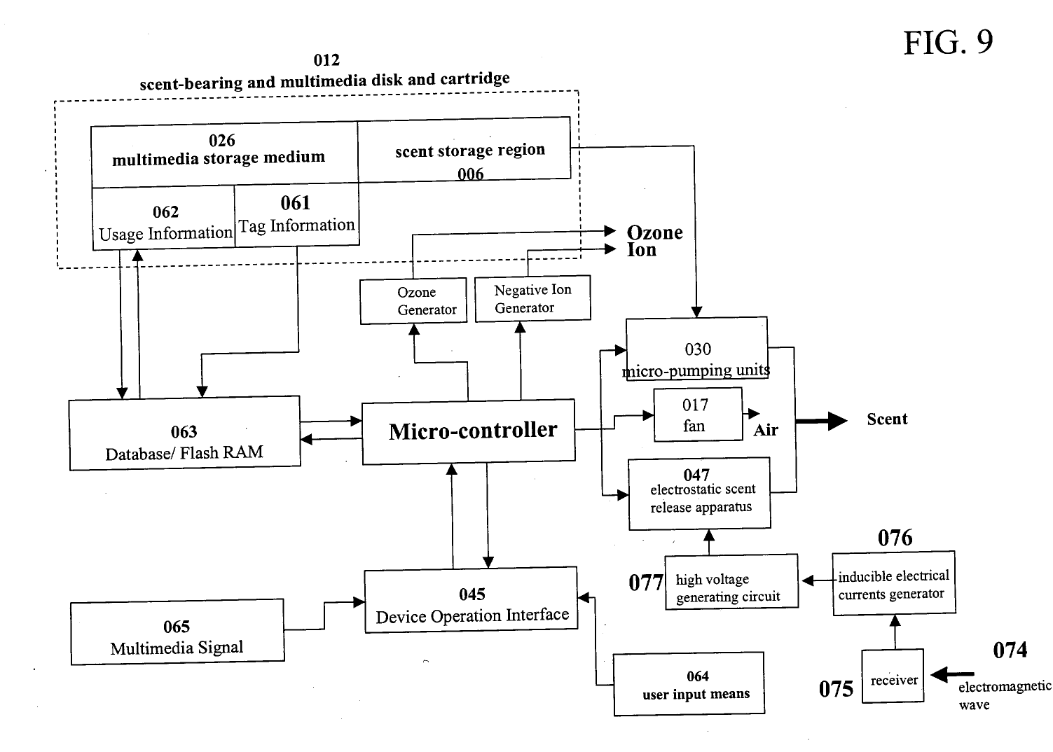

In another preferred embodiment of present invention is depicted in FIG. 9, the electromagnetic wave 074 can be delivered to the receiver 075 of the device and induce electrical currents within the generator 076 which then transform into high voltage by a high voltage generating circuit 077. For advance design, the power supply unit and some control unit can be separated from the scent release unit thus for further wireless operation and device miniaturization.

In addition, we can also use sufficient electromagnetic waves 074 to ionize scents within the scent collecting chamber 010. The ionization process happens like this. The electromagnetic wave entering the scent collecting chamber has energy. As it traverses the scent materials, it interacts with electrons of atoms inside those scent materials. Giving some of its kinetic energy to the electrons, the electromagnetic wave ionizes the scent material, knocking off some electrons from atoms.

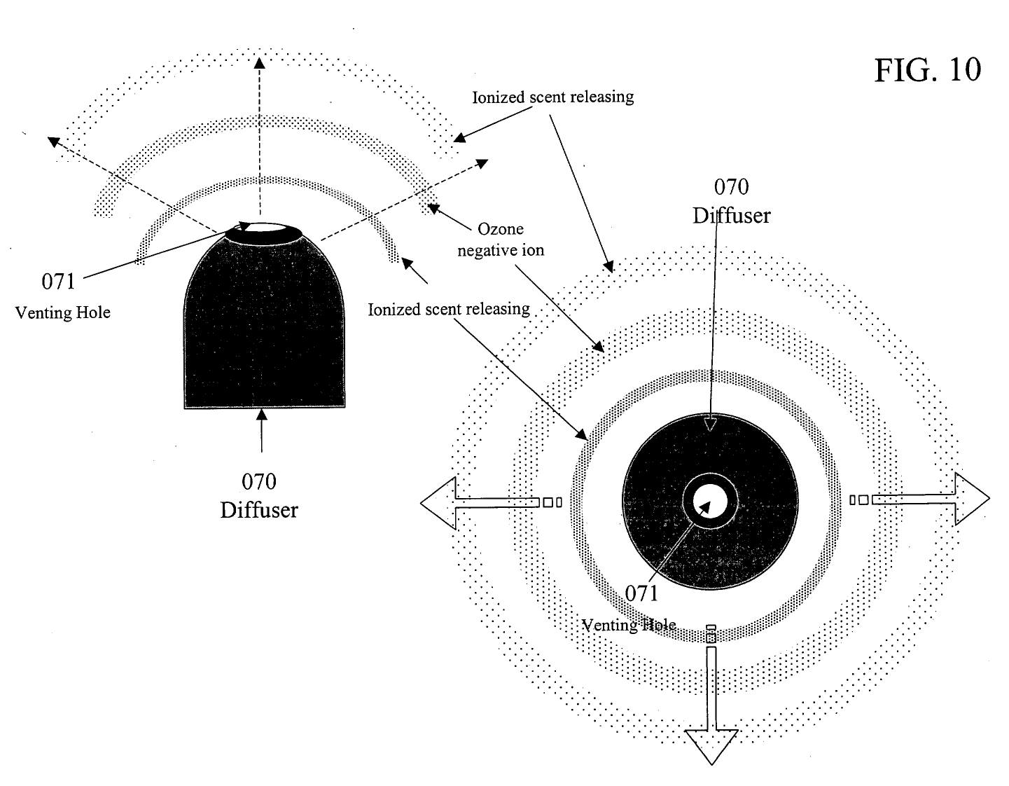

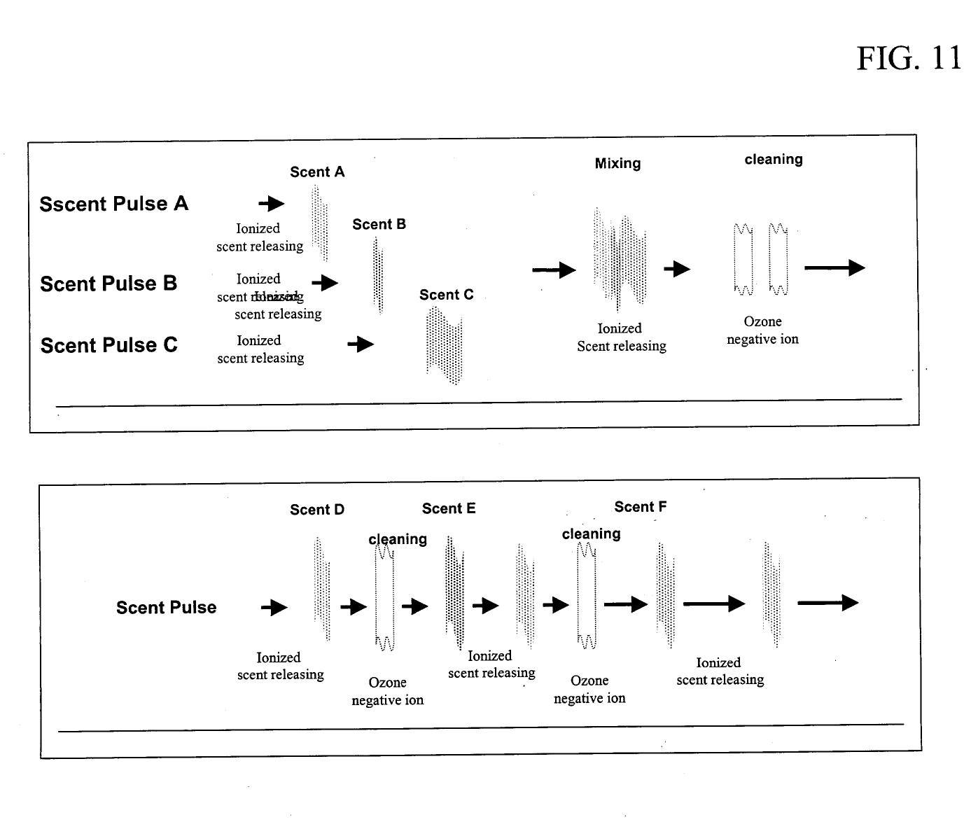

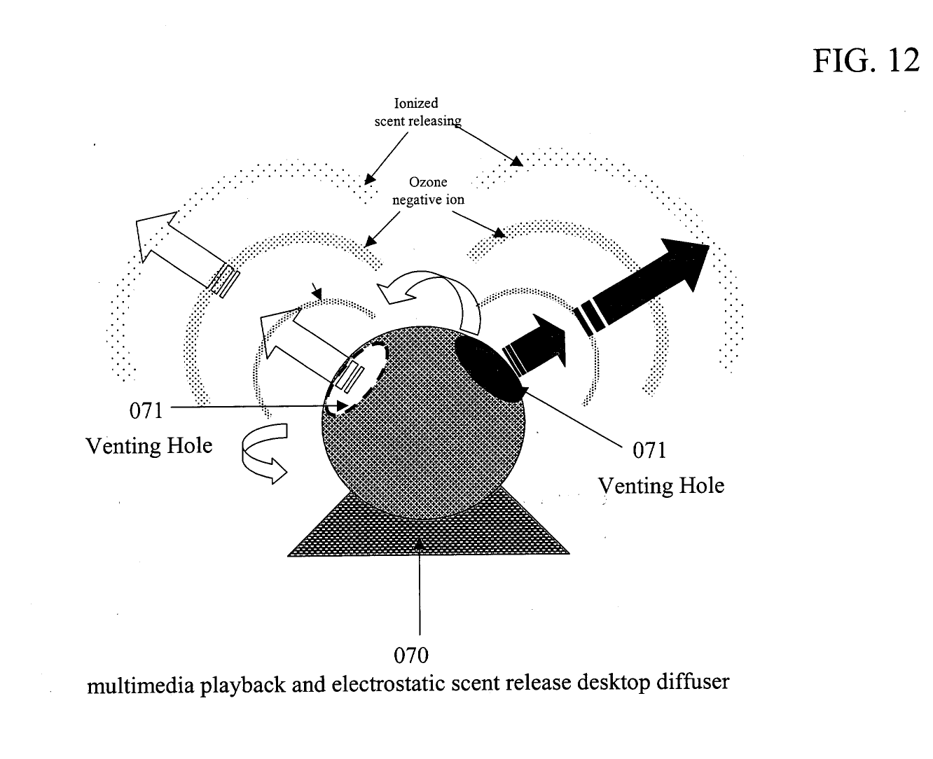

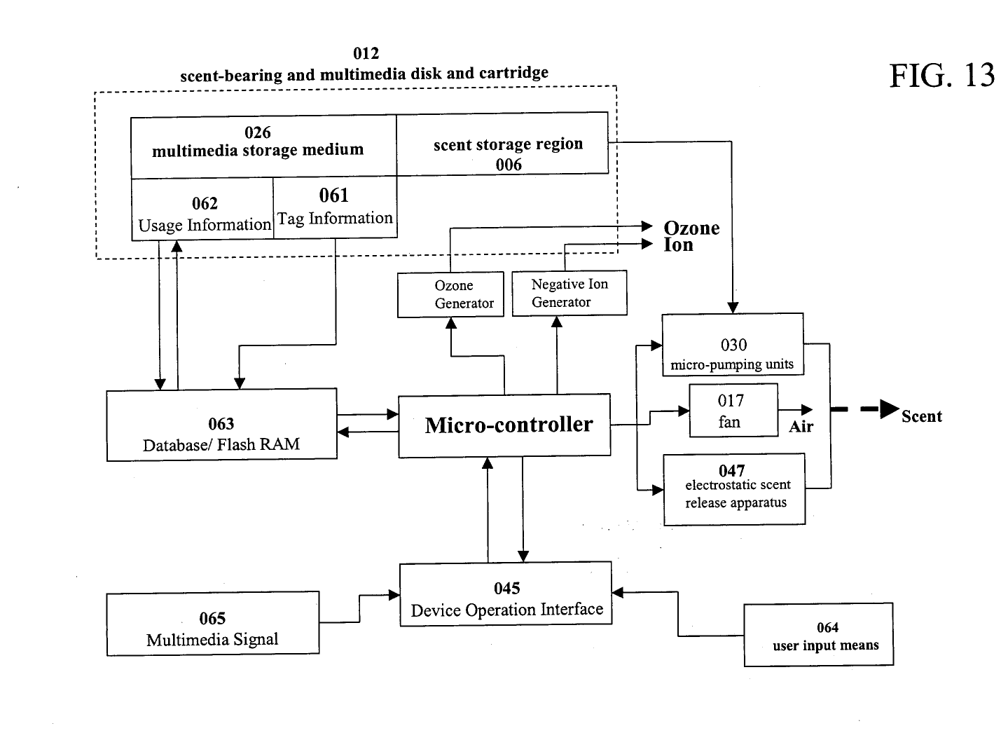

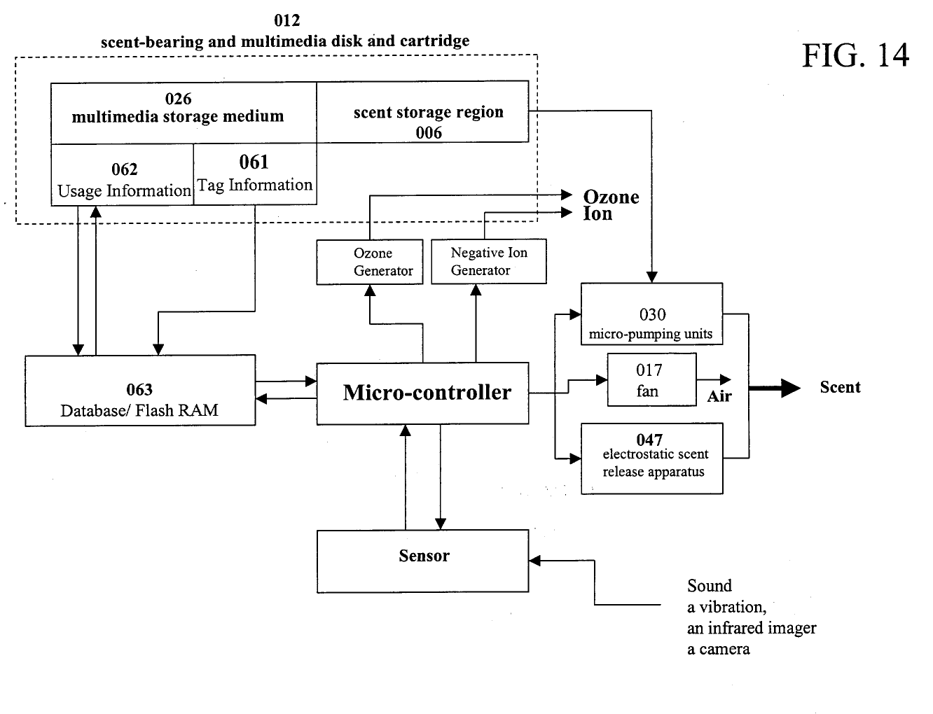

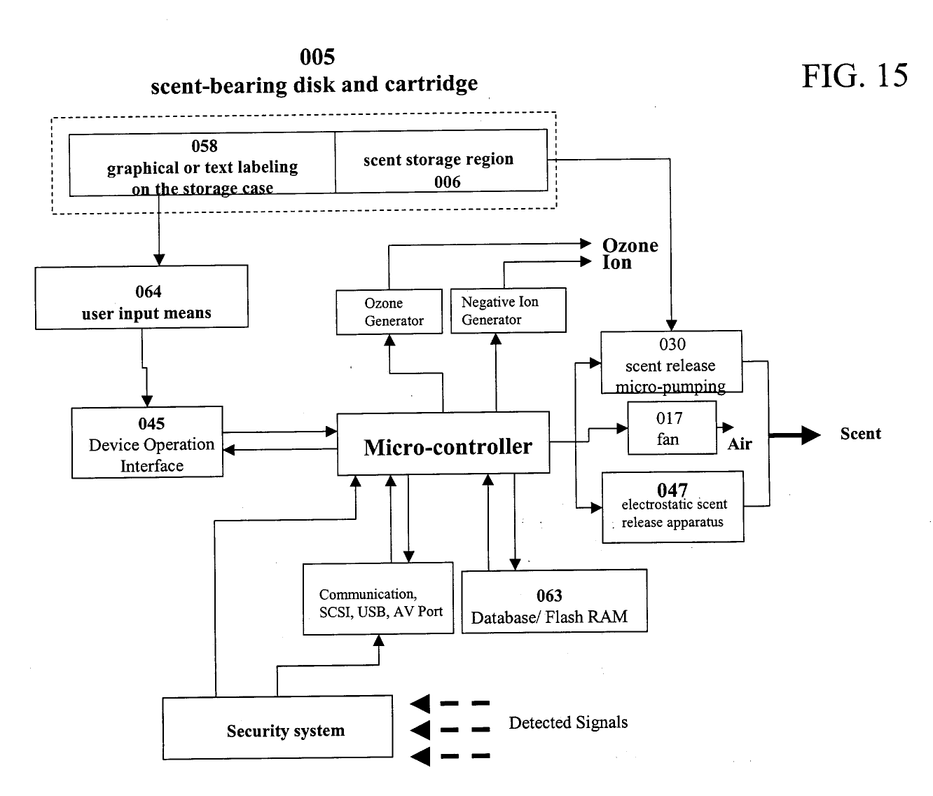

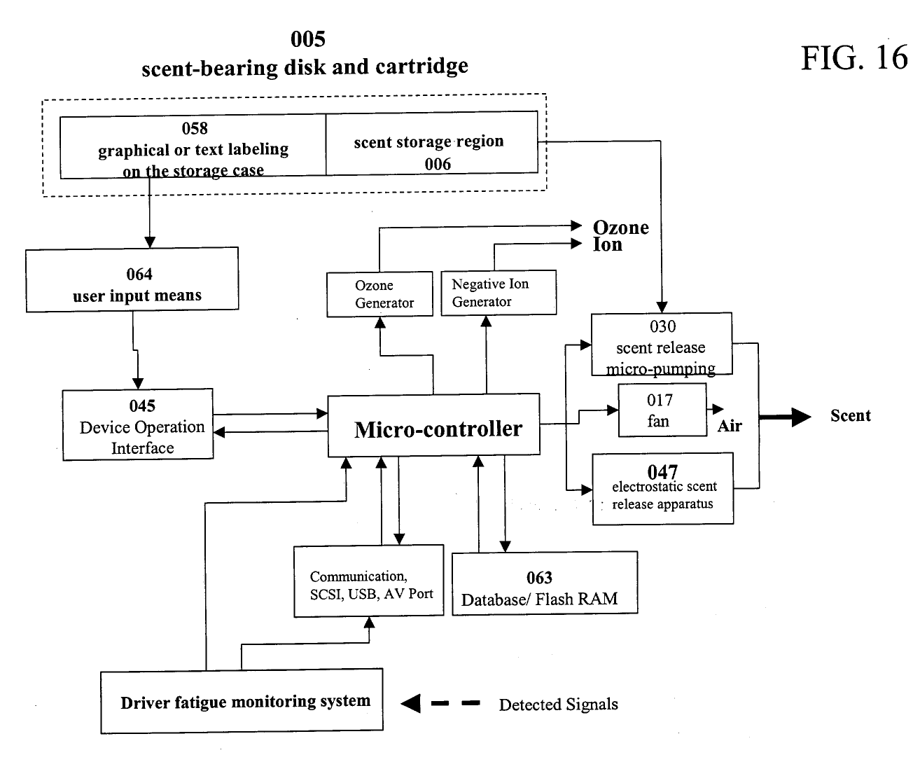

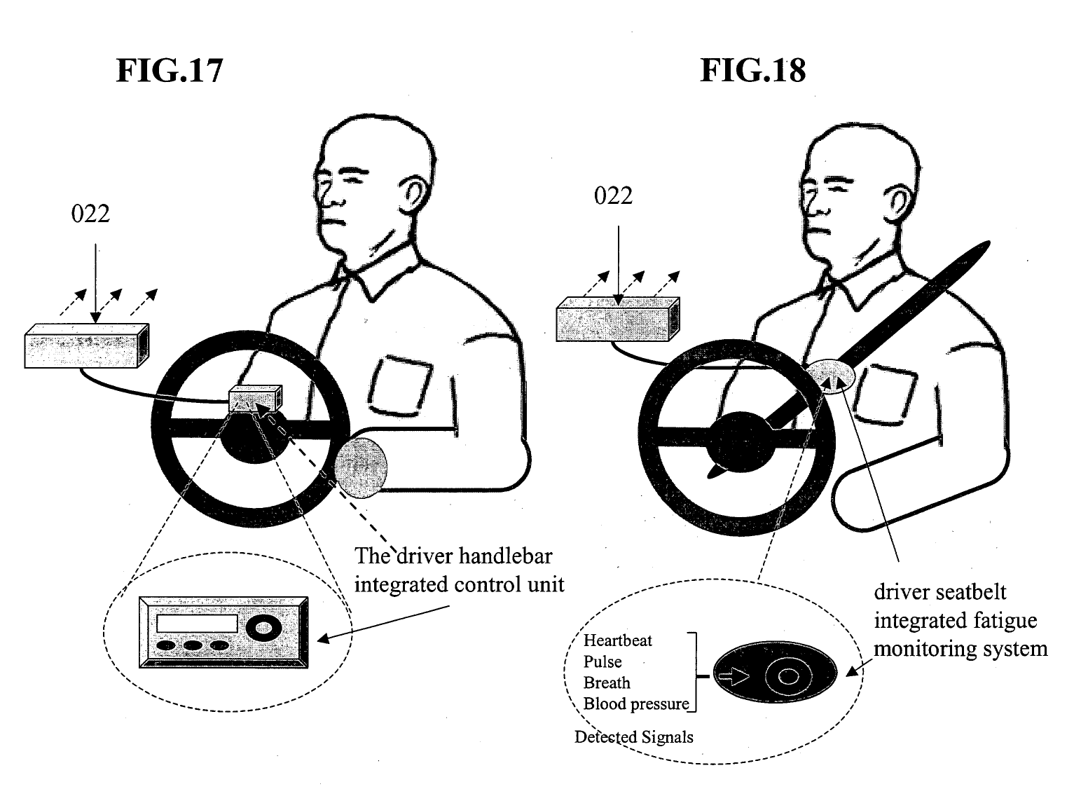

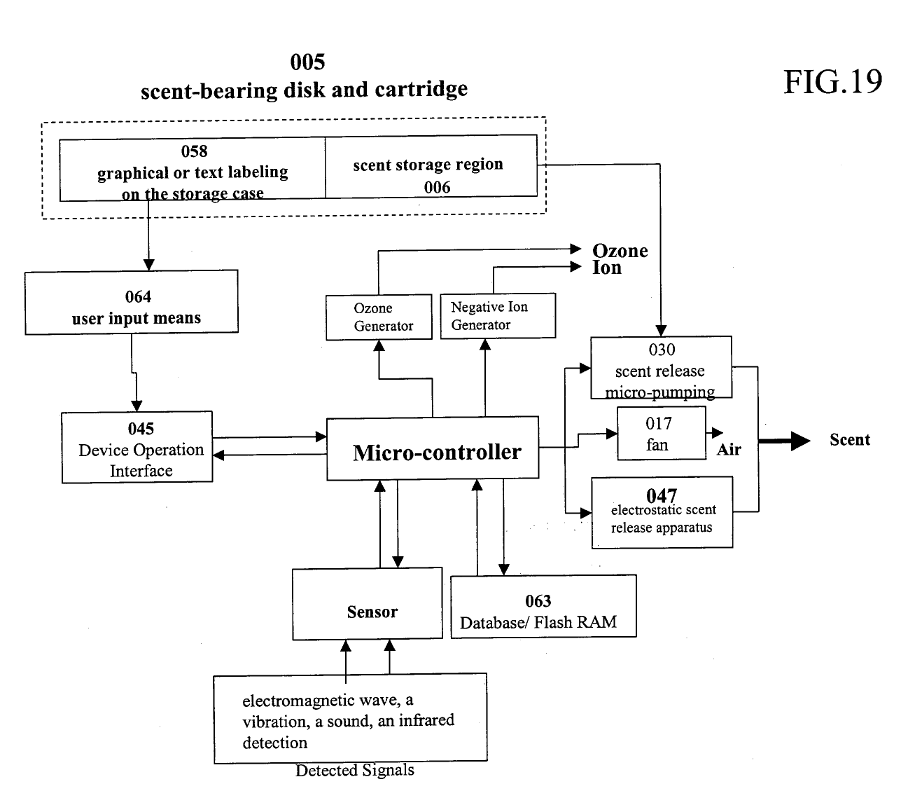

In another preferred embodiment of present invention is depicted in FIGS. 10, 11, 13 and 14, during the scent releasing range control step, a controlled amount of scent neutralizing agent, for example, ozone is released in an air flow faster than the previously-released scent flow to assist in the process of catching up the previously-released scent within a controlled range, thereby neutralizing or degrading the previously-released scent molecules in the air flow; and preventing the scent molecules from trespassing over the controlled range. The step would follow the release scent neutralizing or degrading agent in various angles, as showed in FIG. 12, by automatically rotation of the scent release unit to assist in neutralizing or degrading of the previously-released scent, dust, or other particles within the controlled range.

In another preferred embodiment of present invention is depicted in FIGS. 10, 12 and 14, it will be appreciated however, that the automatically rotation scent releasing may actives in response to a sound, a vibration, an infrared imager a camera, or other means of determining the position of the user and the user's nose.

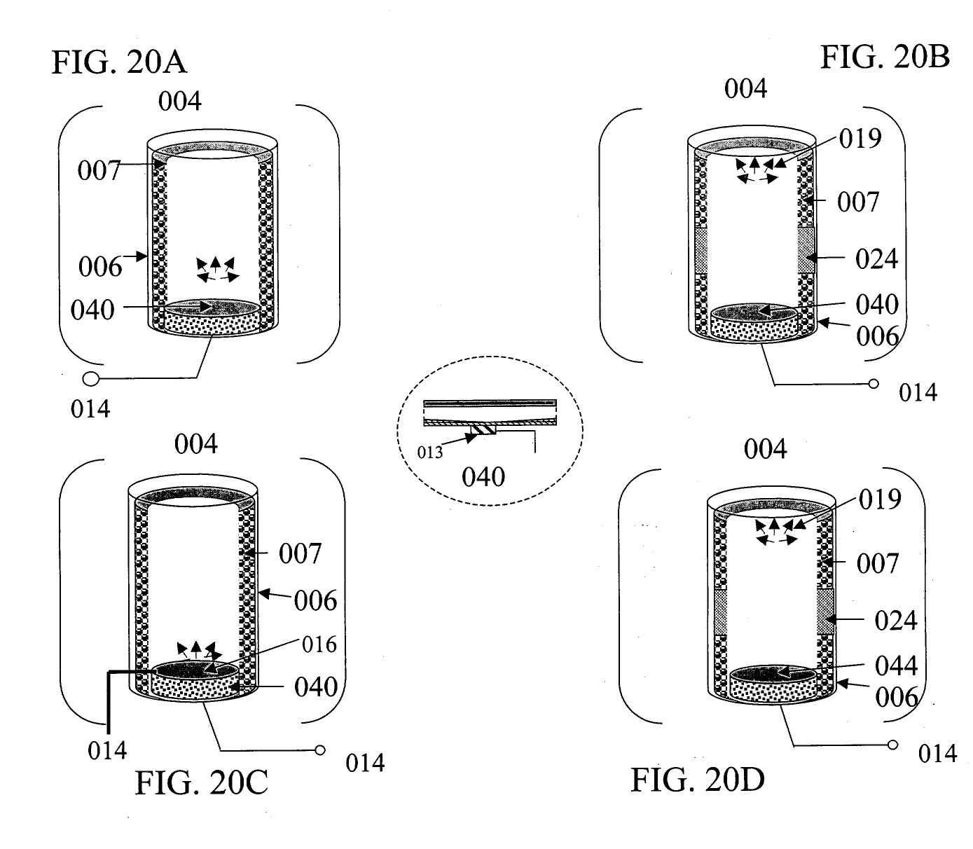

A scent release unit 004 made in accordance with sixth, seventh, and tenth preferred embodiments of the present invention is generally shown in FIGS. 20A-C. The scent release unit 004 comprises an electrically insulated scent storage region 006 containing liquid scent material 007. Contained within the insulated scent storage region 006 is the ultrasonic nebulizing scent release apparatus 040 for nebulizing scent trapped in the scent collecting chamber 010. The piezoelectric vibration plate 013 is electrically connected to a power source by wiring 014. In a variant depicted in FIG. 20B, scent released from scent storage region 006 is released from the scent bearing and multimedia disk by ultrasonic nebulizing scent release apparatus 040. Under the normal air pressure, the multiple-pored-orifice 021 (size less than 80 micron) will be able to retain the scent or drug liquid. When nebulized scent is created within the scent release unit 004, it creates droplet smaller than the pore size and a pressure differential between the region within the scent release unit 004 and immediately outside the orifice. This pressure differential causes the droplet to pass those pores, thereby releasing the scent to the scent venting chamber 011 where it can be vented to the user. In an alternate embodiment shown in FIG. 20B electrostatic electrode 024 may be used to assist in the ionization of nebulized scent vapor. There is another alternate embodiment shown in FIG. 20C, thermal-wiring-integrated orifice 016 may be used to assist in the vaporization and remove retention of scent on the orifice 016. In a further alternate embodiment shown in FIG. 20D electrostatic electrode 024 may be used to assist in the ionization of vaporized scent released from the thermal scent release apparatus 044.

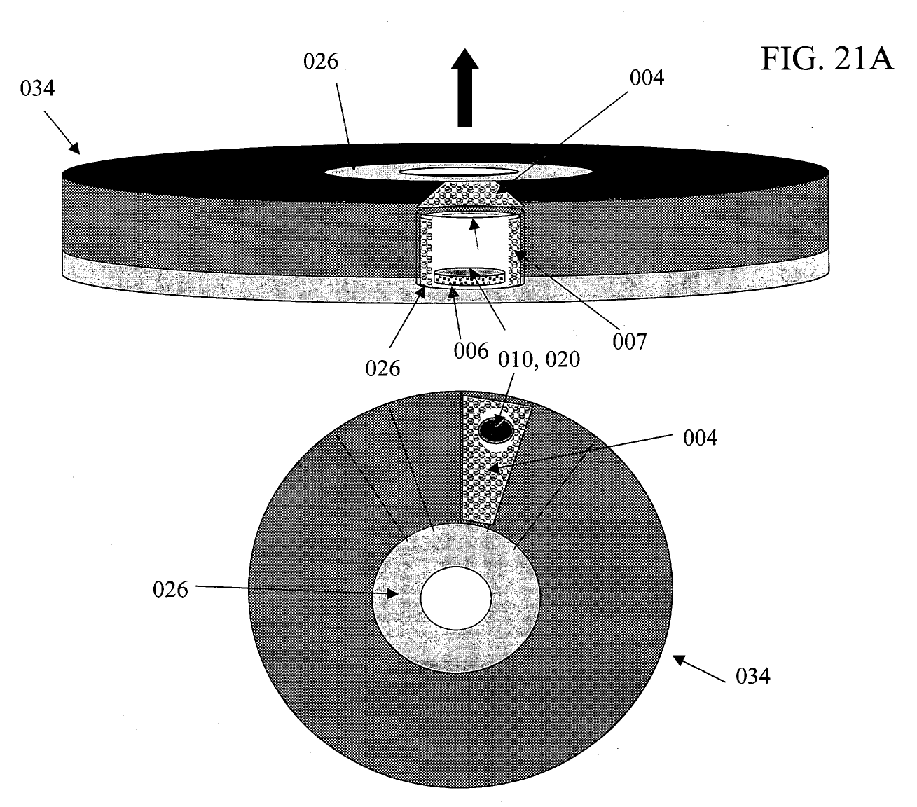

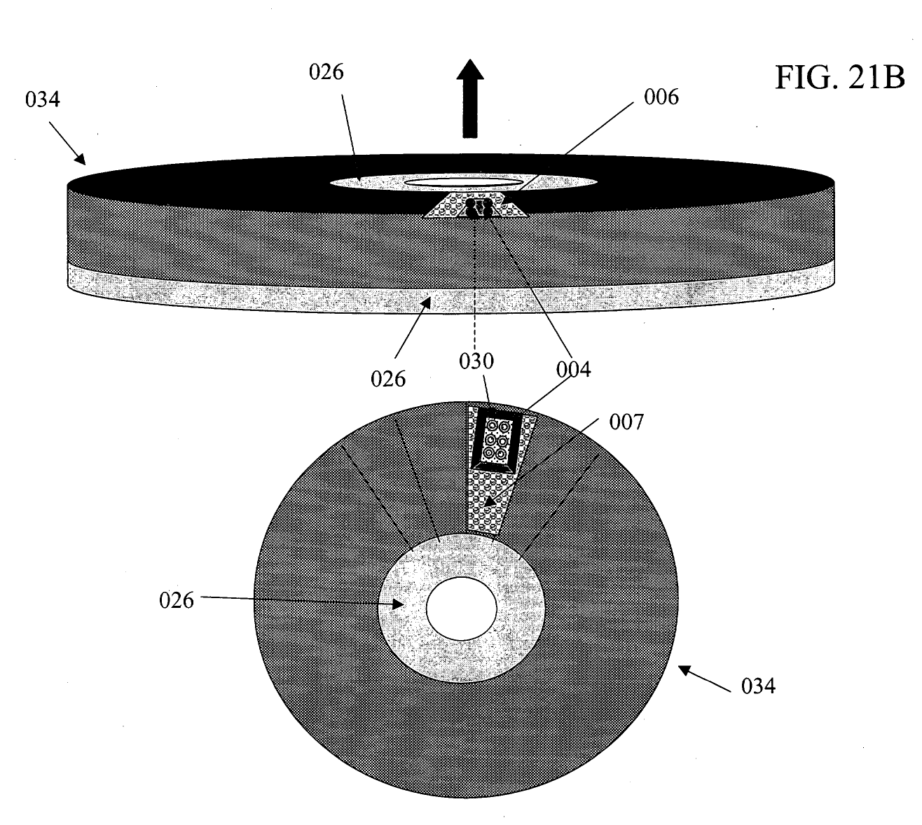

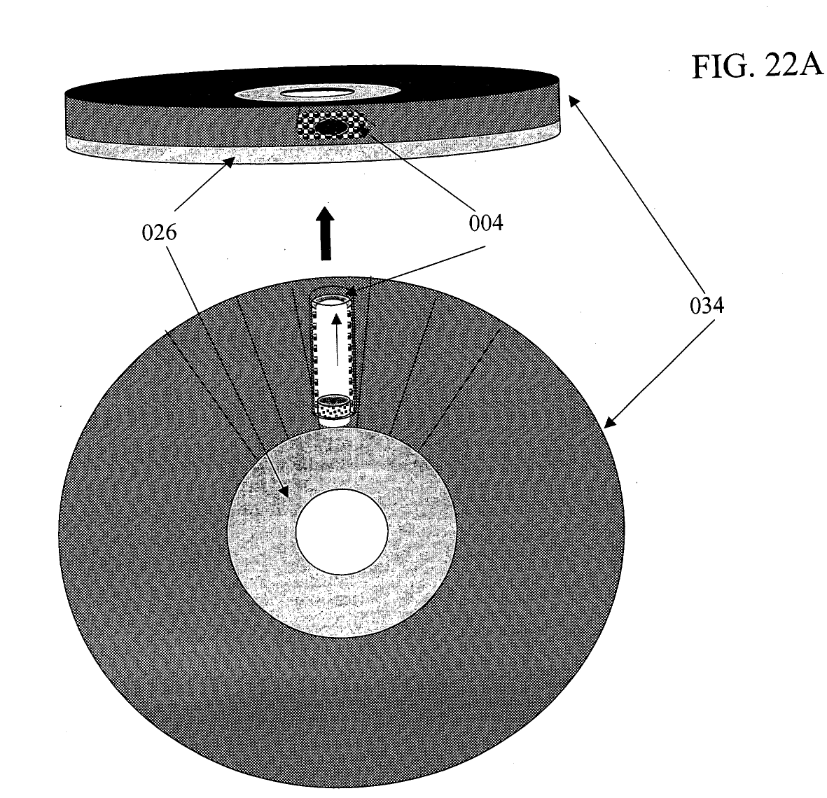

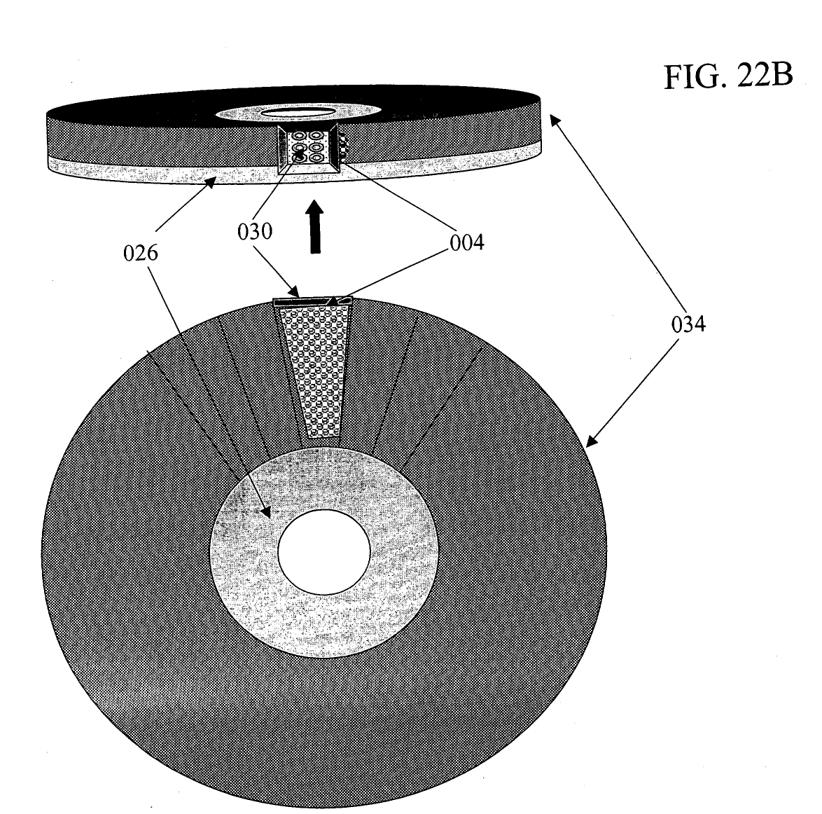

A complete scent-bearing and multimedia disk 034 having scent release unit 004 and multimedia storage medium 026 made in accordance with the tenth preferred embodiment of the present invention is shown in FIGS. 21A and 21B. The scent-bearing and multimedia 034 shown in FIG. 21A comprises a plurality of separate scent or drug release units 004, each of which is contained in a three-dimensional sector of the disk. The scent-bearing and multimedia disk 034 has a multimedia region shown in the interior portion of the disk. The scent-bearing portion of the disk contains a reservoir connected to the portion of the sector containing the scent release units 004. In an alternate embodiment shown in FIG. 21B, the scent-bearing portion of the disk contains at least a reservoir connected to at least a scent release micro-pumping unit 030. In FIGS. 21A and 21B, the scent and multimedia-bearing disk 034 is designed to vent scent vertically upward as indicated by the arrow. In an alternate construction shown in FIGS. 22A and 22B, the scent release units 004 extends laterally along a radius of the disk, thereby permitting the scent or drug to be discharged horizontally through an opening in the circumference of the scent-bearing and multimedia disk 034.

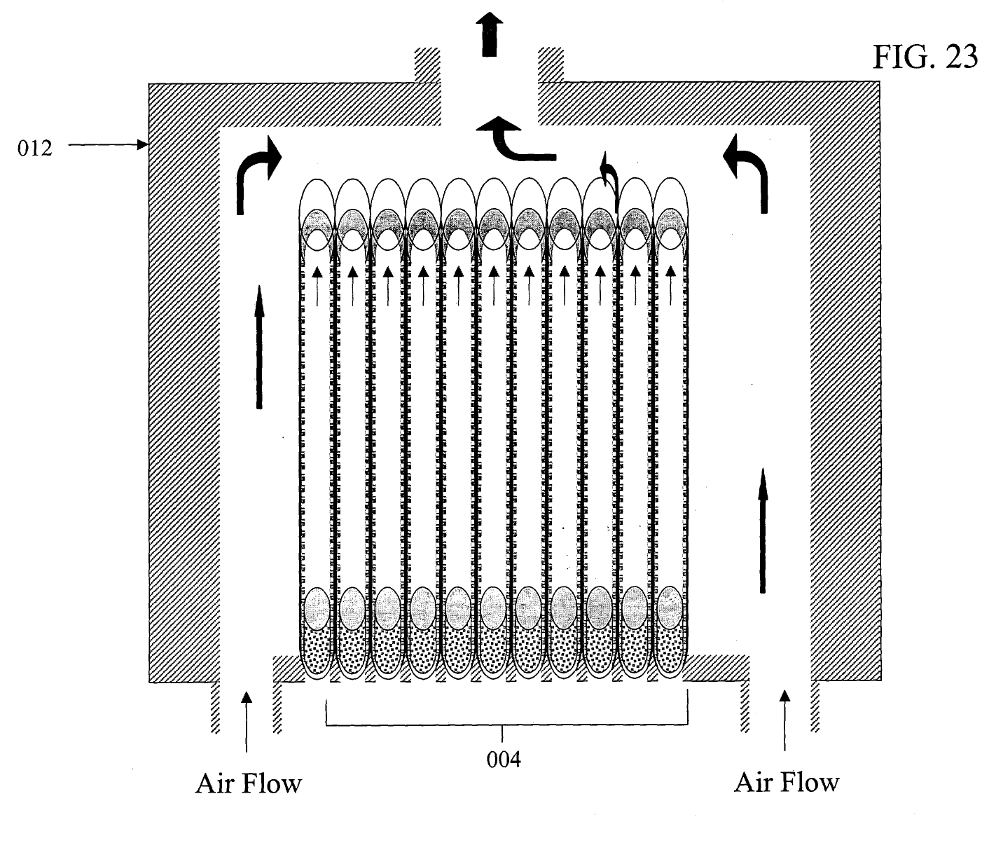

In yet another alternate embodiment suitable for incorporation in a rectangular scent-bearing cartridge, a plurality of scent release units 004 are shown in FIG. 23. The plurality of scent release unit 004 is located in an interior cavity of the scent-bearing and multimedia cartridge 012. The cavity has openings to accept an air flow generated by a fan. The airflow passes by the open end of the scent release units 004. Scent droplet released from the reservoir is entrained in the airflow and vented through opening, where it can be experienced by the user.

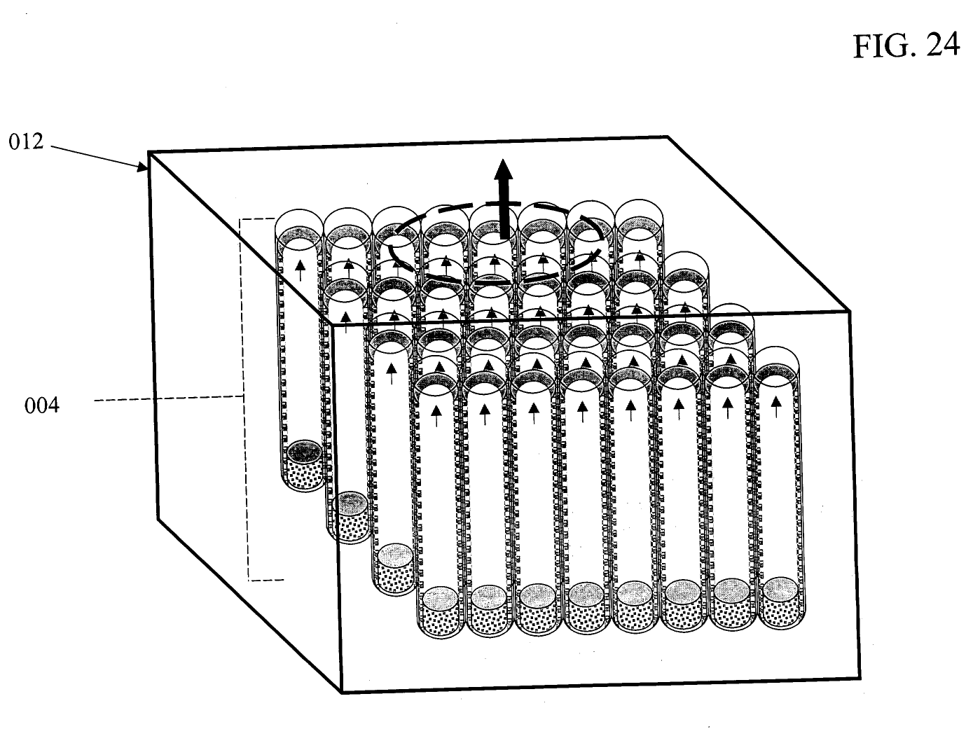

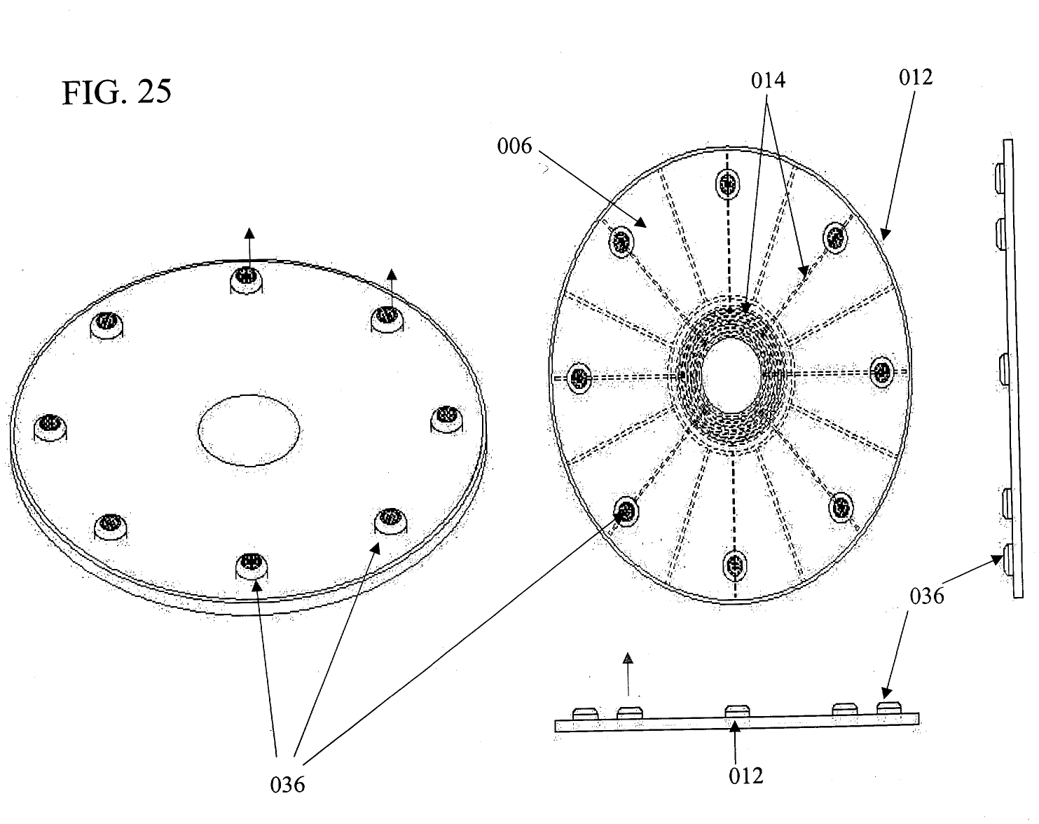

In a still further embodiment of the present invention shown in FIG. 24, a plurality of vertically-oriented scent release units 004 are depicted. In the embodiment of FIG. 25, scent droplet released from the scent release units 004 is vented vertically through single release valves 036.

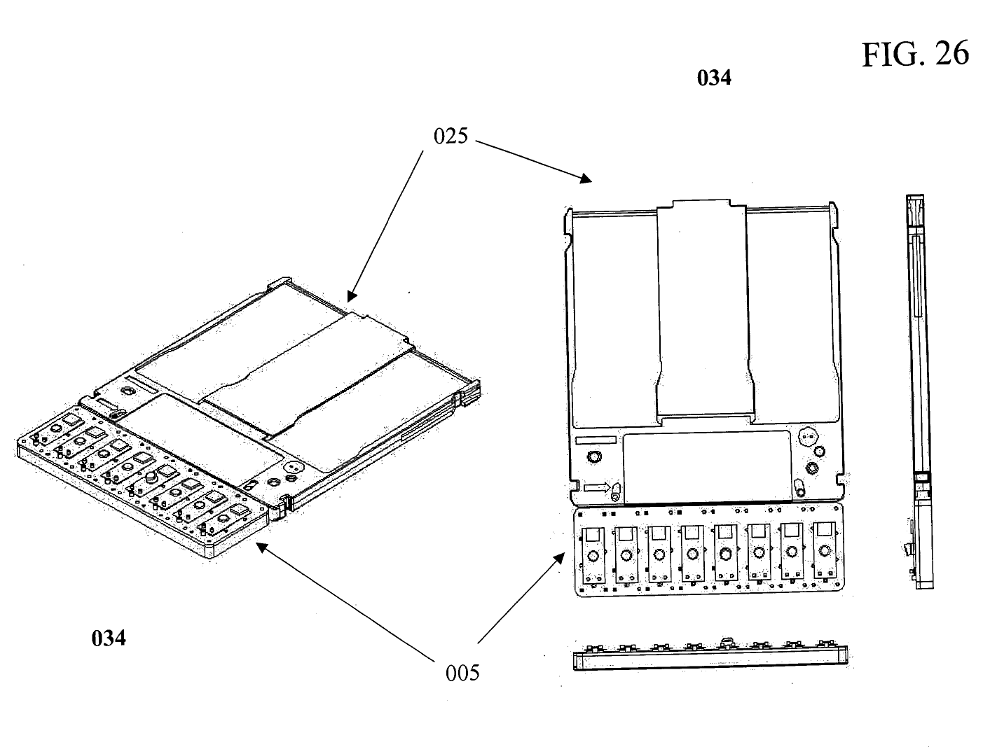

A scent and multimedia -bearing cartridge 012 made in accordance with a tenth preferred embodiment of the present invention is depicted in perspective, top and side views in FIG. 26. The scent and multimedia-bearing cartridge 034 is depicted attached to a Type II DVD-RAM cassette 025 encapsulating an optical storage medium. In variants of the present invention, the encapsulated optical storage media can take the form of DVD-RAM and other known and contemplated DVD storage formats, for example DVDR, DVD-RW, DVD+RW, etc.

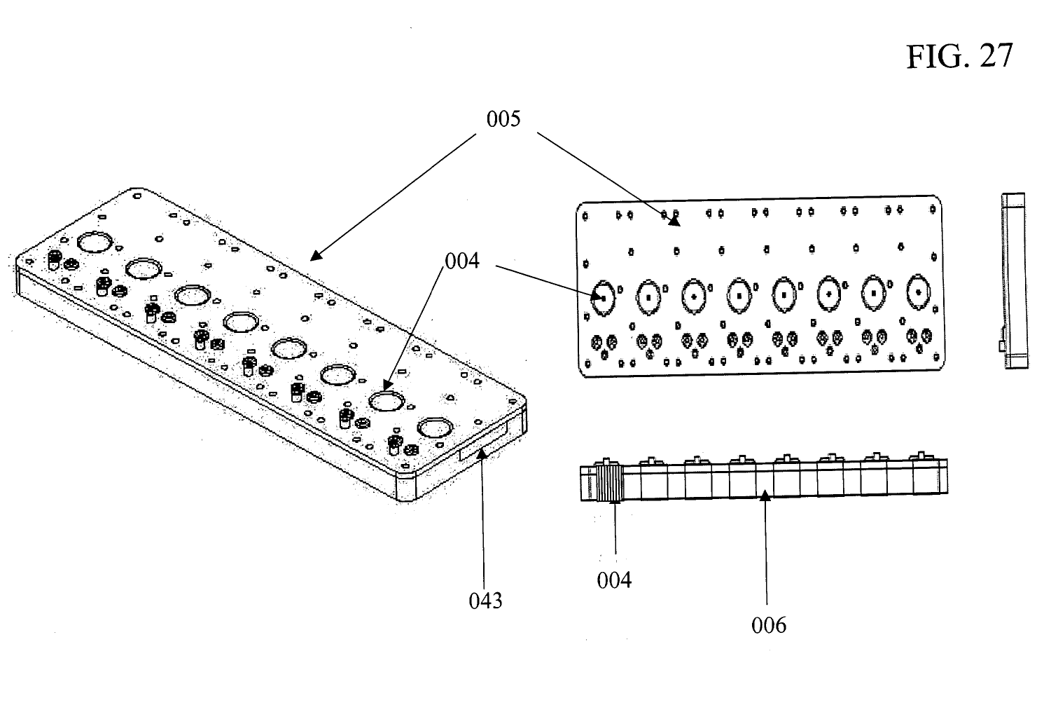

Scent is released from the scent-bearing cartridge 005 using a scent release unit 004 as shown in FIG. 27. The scent release unit comprises at least one scent release unit 004 which deliver scent from the scent storage region 006 to the outlet. In variants of the tenth preferred embodiment, the scent release unit 004 can operate on an evaporation principle, electrostatic principle, or nebulizing principle. The data storage medium 043 (e.g., SIM card, flash memory, memory chip) is shown on the side of scent-bearing cartridge 005.

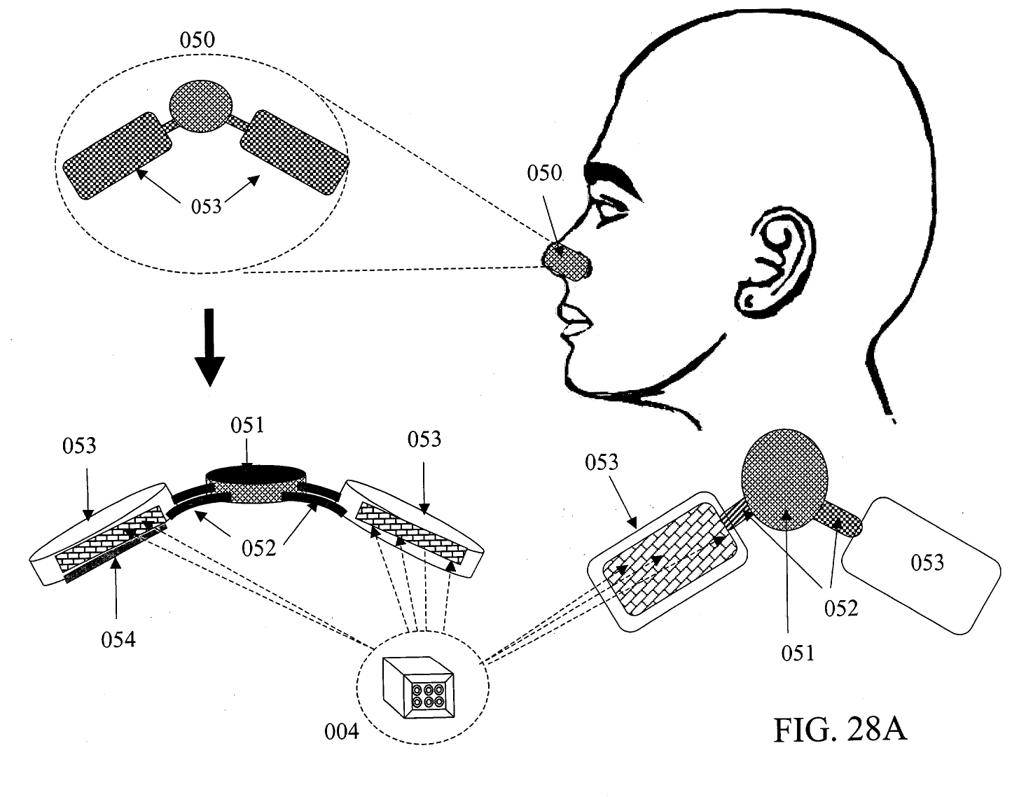

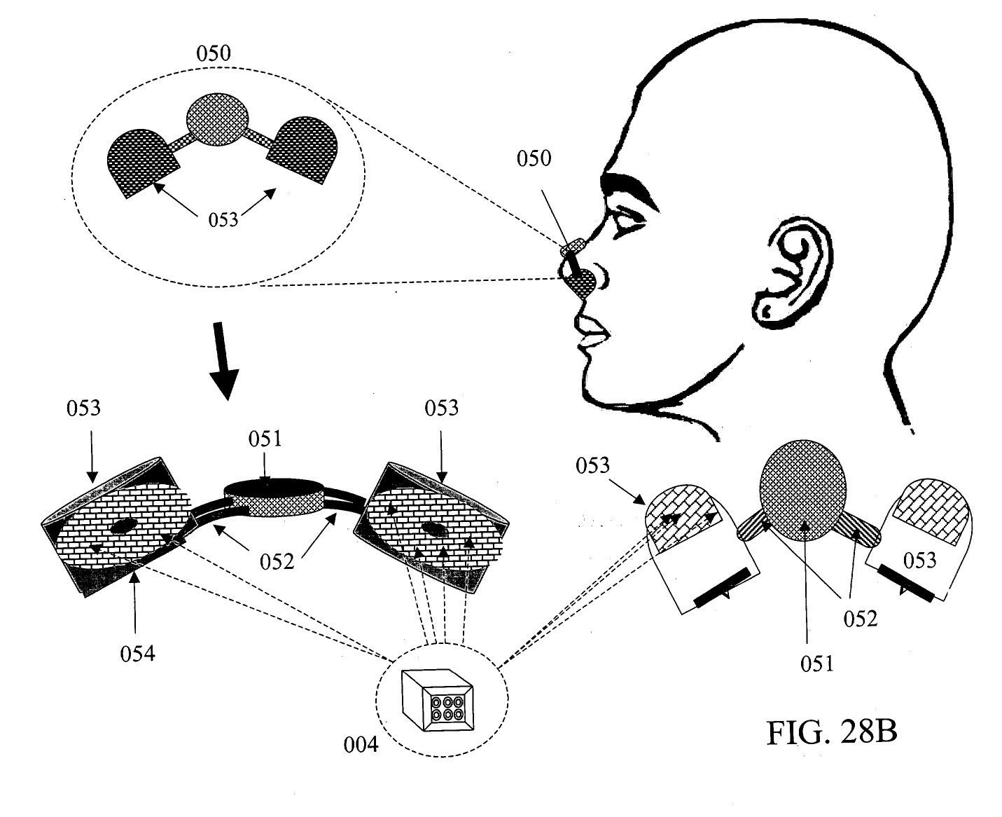

An eleventh preferred embodiment of the present invention is depicted in FIGS. 28A and 28B, and comprises a micro-pump and electrostatic scent release nosepiece 050. The nosepiece 050 is formed of silicon or other material as it may be micro-machined to obtain a very high precision manufacturing and miniaturization. In operation, multiple scents are first stored within the scent storage and micro-pump release unit 053 and controlled by the control unit 051 through the wiring linkage of the flexible plastic piece 052.

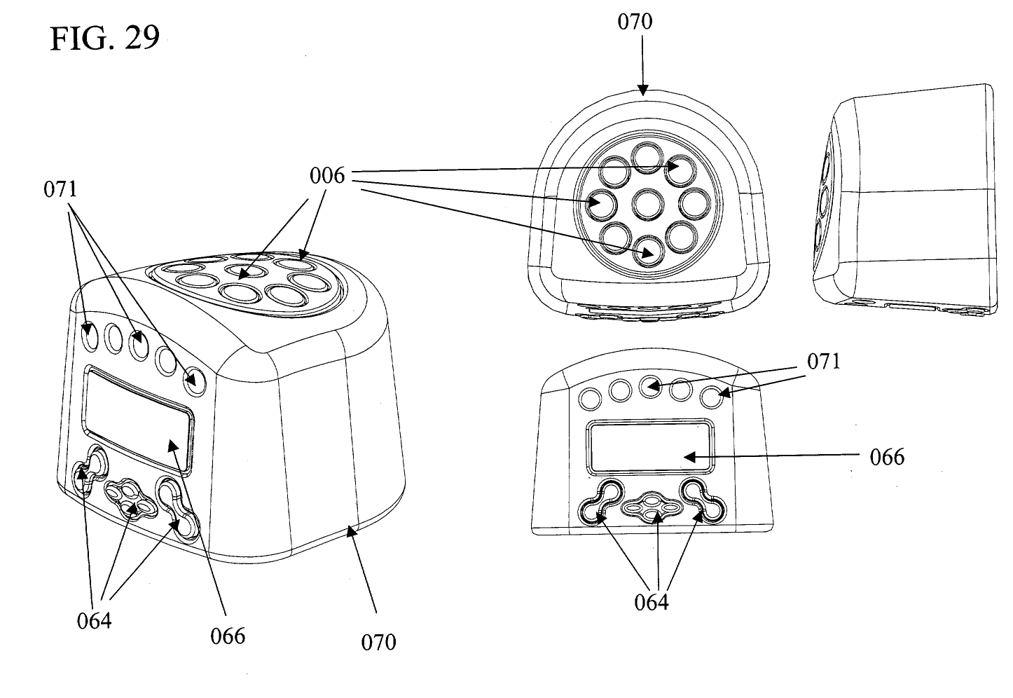

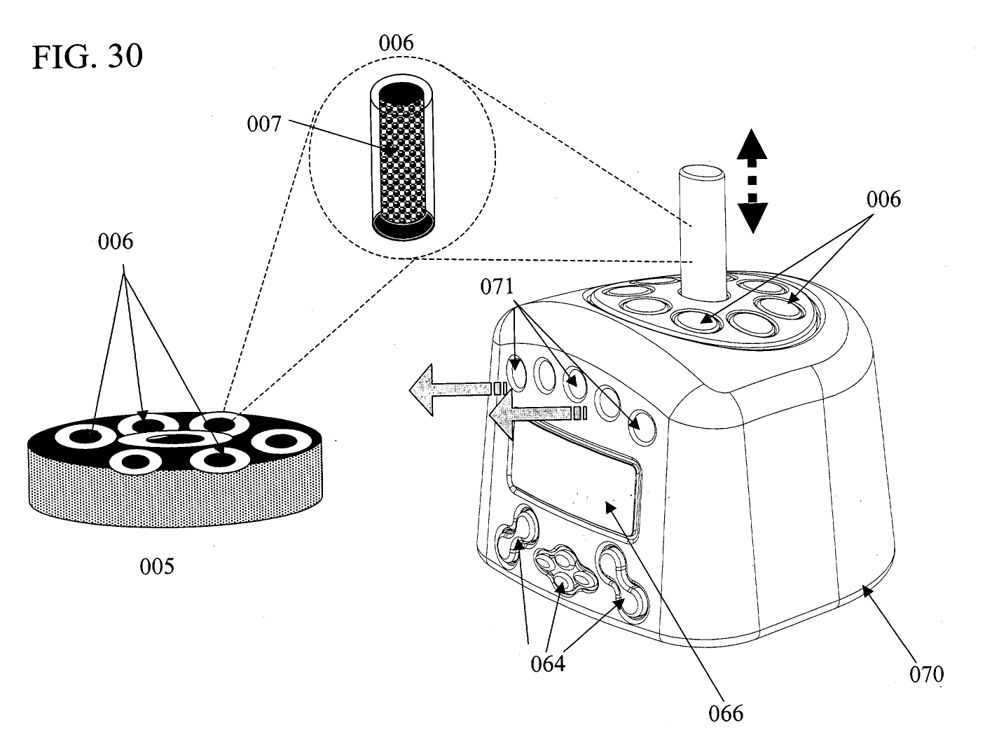

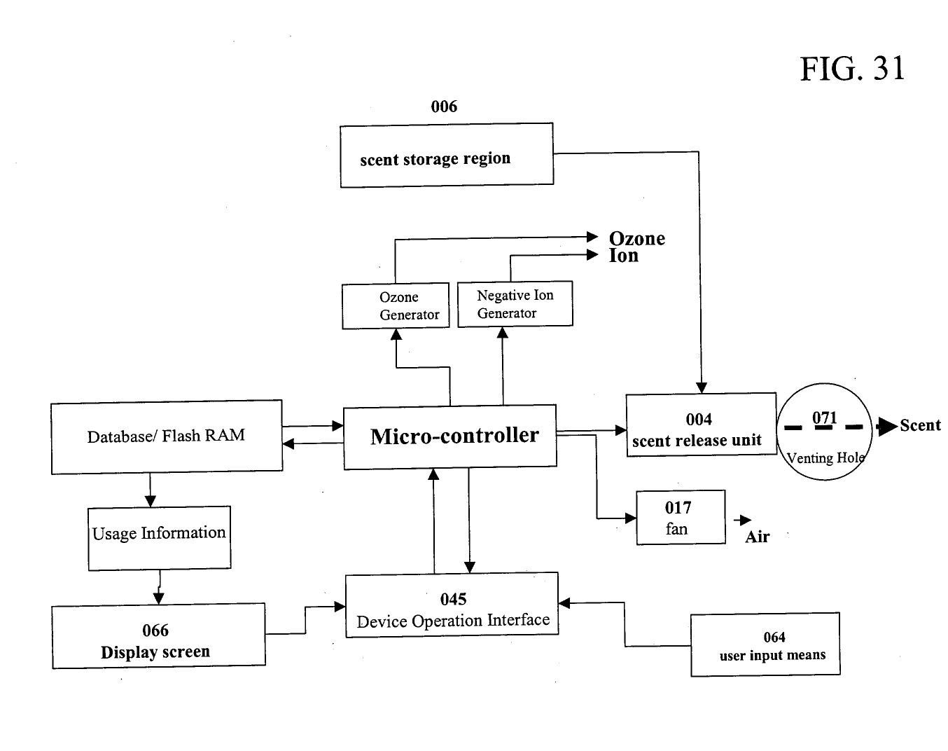

A multimedia playback and electrostatic scent release desktop diffuser 070 having scent-bearing cartridge 005 made in accordance with the twelfth preferred embodiment of the present invention is shown in FIGS. 29, 30 and 31. The multimedia playback and electrostatic scent release desktop diffuser 070 shown in FIGS. 29, 30 and 31 comprises a display screen 066 mounted on the housing to display multimedia information, a housing for containing scent-bearing cartridge 005, speakers to play back auditory information, a user input pad and interface 064, an electrostatic scent release unit, and venting hole 071 for delivering scent into air.

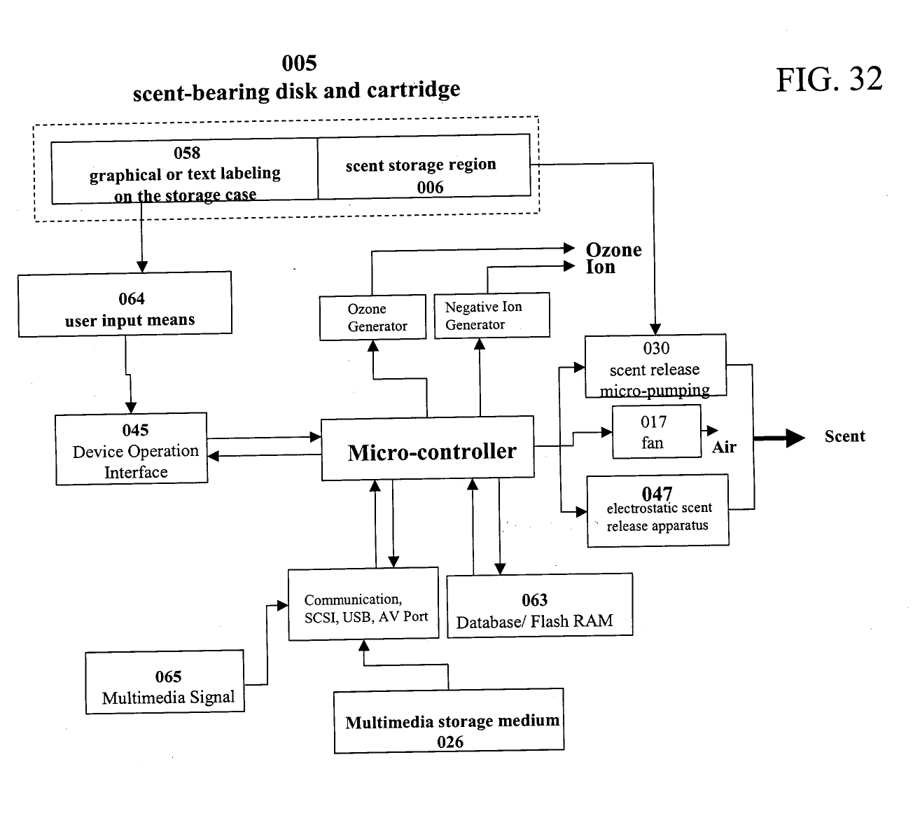

In another preferred embodiment of present invention is depicted in FIG. 32, scent-bearing cartridge 005 has graphical or text label 058 on the case. The content of the graphical or text label will provide the user with scent content information, texts or numbers for inputting into the device, or scent sequence information. The scent-bearing cartridge 005 can be related to the diffuser 070 in a manner which is not dependent on some other multimedia and scent information, and/or are not designed to be used for automatically scent content information downloading with the diffuser. The method for applying user input 064 can through voice activation, infrared or wireless remote control etc. By matching the user input information 064 with the database within the diffuser, the device could recognize the scent-bearing cartridge and operate properly.

The scents can be related to each other in a manner which is not dependent on some other media, and/or are not designed to be used simultaneously with other media for the relationship between the scents. In addition user can customize the selection and arrangement of scents for an automatic multiple scents aromatherapy.

In another variant of present invention, the diffuser can be provided with a remote control so that the diffuser can be operated from a distance. The diffuser can also be provided with a timer that will allow the user to program the diffuser to start at a particular time. Preferably, in such an embodiment, the timer is integral with, and built into the diffuser. In other embodiments, a separate timer can be used with the diffuser.

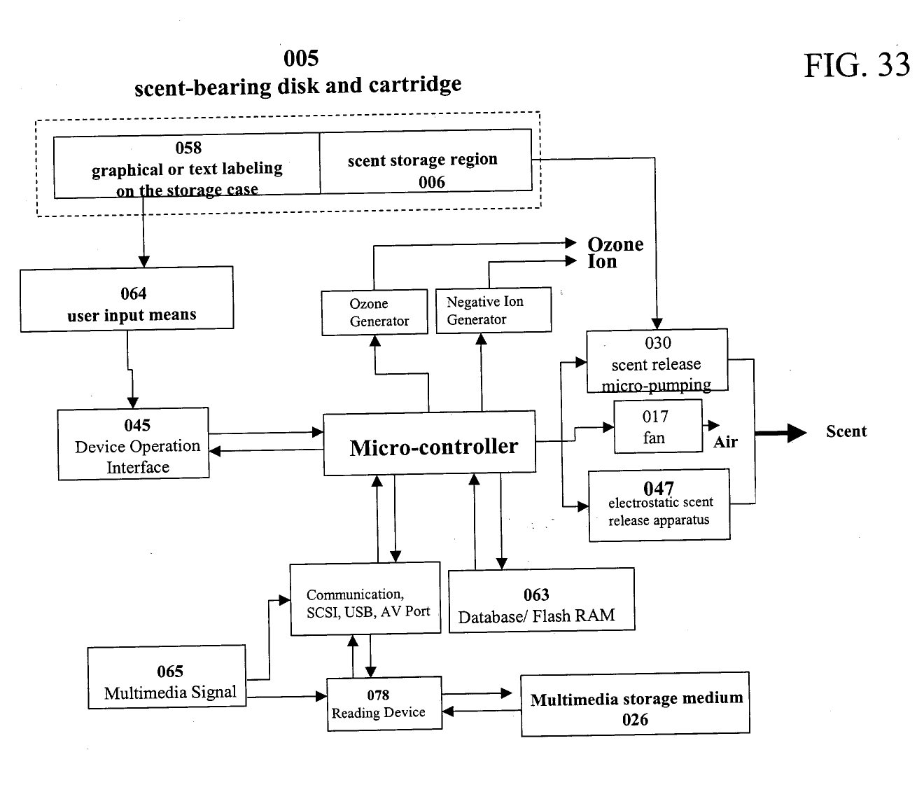

In another preferred embodiment of present invention is depicted in FIG. 33, a scent-bearing cartridge 005 has graphical or text label 058 on the case. The scent content information of the scent-bearing cartridge 005 can be provided by a separate multimedia storage medium 026 which can be download into the diffuser by a build-in or linked reading device 078 which connecting to the diffuser directly or indirectly. The scent-bearing cartridge 005 can be related to the scent content information in a manner which is not directly integrated with some other multimedia and scent information, and/or are not designed to be used for automatically scent content information downloading with the diffuser.

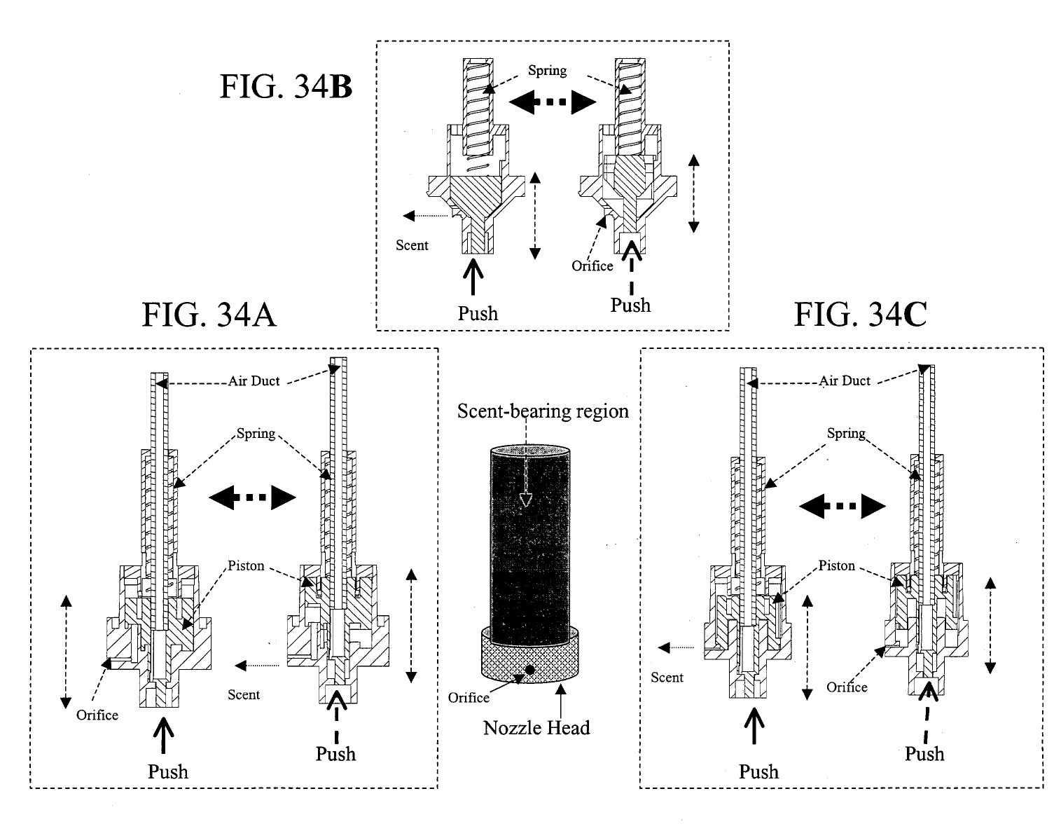

In another preferred embodiment of present invention is depicted in FIGS. 34A, 34B, and 34C, a special design nozzle head for pumping scent through the activation of a solenoid during scent releasing operation to improve the flow of scent from the scent storage region to the scent release unit.

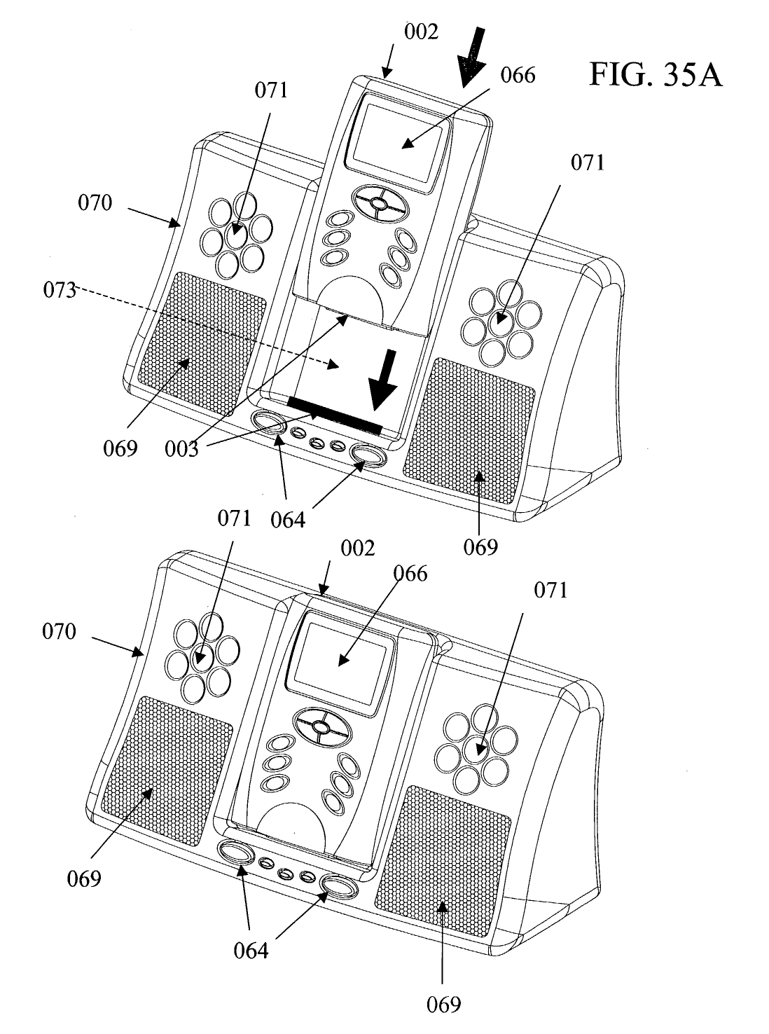

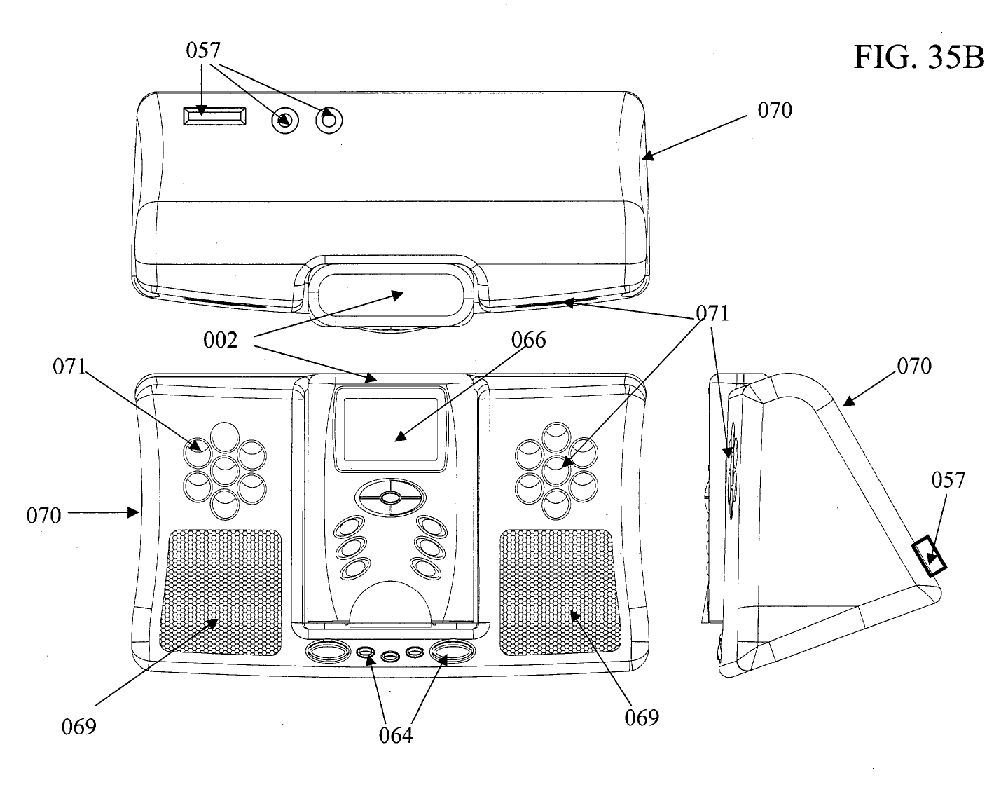

In another preferred embodiment of present invention is depicted in FIGS. 35A and 35B, a hand-held device and a alternate multimedia playback and electrostatic scent release desktop diffuser 070 has a communication and power supply port 003 can connect with the device interface of a hand-held device 002. The alternate multimedia playback and electrostatic scent release desktop diffuser 070 contains a docking structure for holding a hand-held device, a multiple scent release units, a electrostatic scent release apparatus 047, a user input means 064, and speakers 069. In addition, the communication and connection port 057 (e.g. SCSI, USB, or AV port) can link to other electronic device. The hand-held device 002 can provide as a wireless communication between a remote signal source and the controller of the alternate multimedia playback and electrostatic scent release desktop diffuser 070 for playing audio, video, text, graphs, or scents. In a further advantage, the alternate multimedia playback and electrostatic scent release desktop diffuser 070 can provide a power recharging operation for the hand-held device 002 through the communication and power supply port 003.

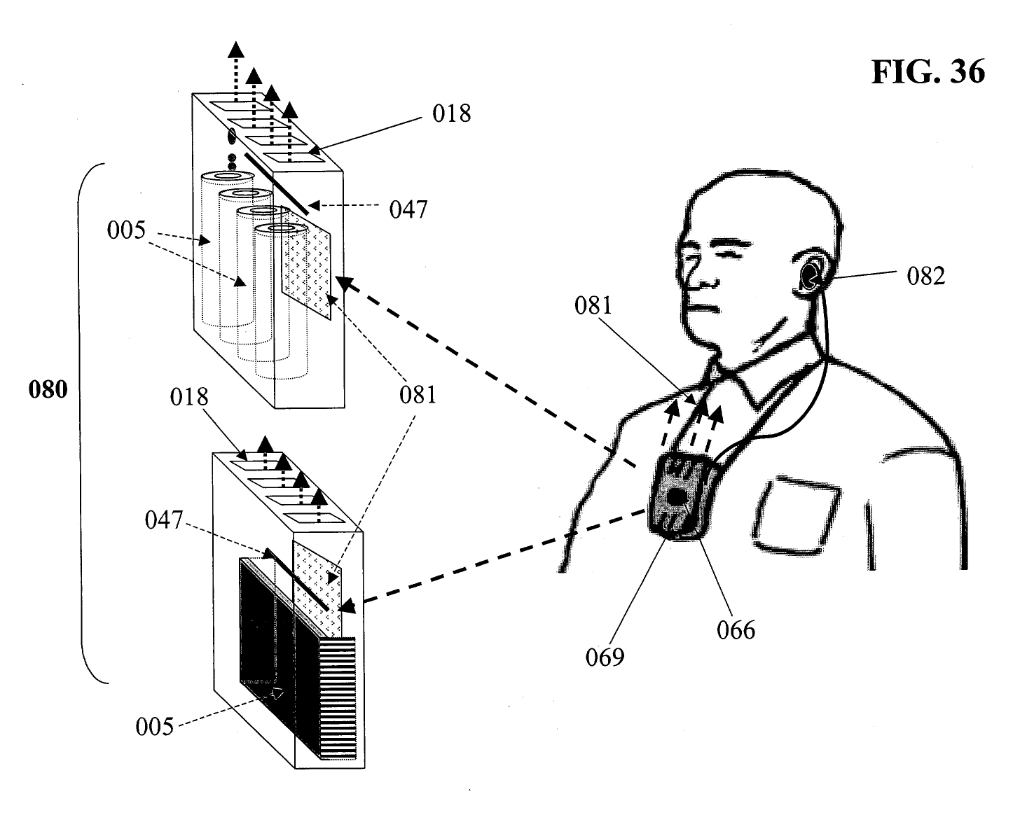

In another preferred embodiment of present invention is depicted in FIG. 36, a multimedia playback and electrostatic scent release personal diffuser 080 comprises an attachment means 081 placed directly adjacent to the housing of the diffuser, a display screen 066 mounted on the housing to display multimedia information, an earphone port to output auditory information to earphone 082, a housing for containing scent-bearing cartridge 005, speakers 069 to play back auditory information, a user input pad and interface and an electrostatic scent release apparatus 047. The attachment means 081 comprises a clip structure, a drawstring, an interlocking, a loop of fabric, a VELCRO-like fabric patches, or a mechanical snap. Electrostatic scent release apparatus comprise at least an electrostatic discharge needle and associated wiring for ionizing scent, fragrance, aroma, therapeutic agent, or flavor. A fan and ducting to create an air flow in the vicinity of the electrostatic discharge needle to assist in entraining scent ionized by the electrostatic discharge needle. Electrostatic scent release units included in variants of the fifteenth embodiment further comprise electrostatic grids 018 for attracting ionized scent molecules to improve efficiency of scent released.

1. A scent-bearing disk or cartridge for use with encapsulated multimedia storage devices of the type wherein a multimedia storage medium is contained in a storage housing, the scent-bearing disk or cartridge and encapsulated multimedia storage devices for use with a separate multimedia playback and scent recovery/release system, the scent-bearing disk or cartridge comprising:
a scent storage region for storing at least one scent;
a scent release unit;
a scent conducting tube connecting the scent storage region to the scent release unit, wherein during scent release operations scent flows from the scent storage region to the scent release unit; and
an encapsulated multimedia storage medium for storing multimedia and scent release control information.

2. The scent-bearing disk or cartridge of claim 1 wherein the scent storage structure integrates with a wireless communication device or a personal assistance device, the hand-held device integrated scent-bearing disk or cartridge having a housing structure encapsulating a removable multiple scent storage region and a communication port with the hand-held device which it receive the control signal from the hand-held device.

3. The scent-bearing disk or cartridge of claim 1 wherein the scent storage region stores a plurality of scents.

4. The scent-bearing disk or cartridge of claim 1 wherein the scent storage region comprises:
a housing for containing scent storage structures and scent release micro-pumping units; and
a scent storage reservoir contained in the housing.

5. The scent-bearing disk or cartridge of claim 4 wherein the scent release micro-pumping unit comprises:
a nozzle plate for pumping scent through scent conducting tube during scent release operations to improve the flow of scent from the scent storage region to the scent release unit.

6. The scent-bearing disk or cartridge of claim 5 wherein the nozzle plate comprises a three dimensional region enclosed on all sides except having a scent release opening facing scent release unit for releasing scent from the scent storage region, and a scent conducting tube connecting the scent storage region to the scent release unit; and
scent pumping means for pumping scent through scent conducting tube during scent release operations to improve the flow of scent from the scent storage region to the scent release unit.

7. The scent-bearing disk or cartridge of claim 1 wherein the scent release unit is made from anti-erosive material and wherein the scent release unit further comprises:
a scent collecting chamber for collecting and then releasing scent into air; and
scent releasing means for releasing the scent into the air.

8. The scent-bearing disk or cartridge of claim 6 wherein the scent pumping means further comprises:
piezoelectric scent pumping apparatus for pumping scent through scent conducting tube during scent release operations to improve the flow of scent from the scent storage region to the scent release unit.

9. The scent-bearing disk or cartridge of claim 6 wherein the scent pumping means further comprises:
thermal bubble scent pumping apparatus for pumping scent through scent conducting tube during scent release operations to improve the flow of scent from the scent storage region to the scent release unit.

10. The scent-bearing disk or cartridge of claim 6 wherein the scent pumping means further comprises:
micro-valve scent pumping apparatus for conducting scent through scent conducting tube during scent release operations to improve the flow of scent from the scent storage region to the scent release unit.

11. The scent-bearing disk or cartridge of claim 6 wherein the scent pumping means further comprises:
electrophoresis scent pumping apparatus for conducting scent through scent conducting tube during scent release operations to improve the flow of scent from the scent storage region to the scent release unit.

12. The scent-bearing disk or cartridge of claim 5 wherein the nozzle plate and the scent storage region further comprises:
a integrated nozzle plate and scent storage reservoir unit.

13. The scent-bearing disk or cartridge of claim 5 wherein the nozzle plate and the scent storage region further comprises:
a detachable nozzle plate and scent storage reservoir unit.

14. The scent-bearing disk or cartridge of claim 7 wherein the scent releasing means further comprises:
electrostatic scent release apparatus for ionizing scent in the scent collecting chamber.

15. The scent-bearing disk or cartridge of claim 7 wherein the scent releasing means further comprises:
ultrasonic nebulizing scent release apparatus for vibrating scent in the scent collecting chamber.

16. The scent-bearing disk or cartridge of claim 7 wherein the scent releasing means further comprises:
thermal scent release apparatus for vaporizing scent in the scent collecting chamber.

17. The scent-bearing disk or cartridge of 7 wherein the scent collecting chamber further comprises:
a groove structure on the bottom surface of the scent collecting chamber to divide multiple scent onto each individual collecting zone, whereby the groove structure hinder one scent from trespassing onto another collecting zone while scent is releasing into the scent collecting chamber.

18. The scent-bearing disk or cartridge of 7 wherein the scent collecting chamber further comprises:
a orifice mounted above the piezoelectric vibration plate of the scent collecting chamber to assist in vaporizing scent into air through the pores of the top orifice, whereby, the orifice with multiple pore (size less than 80 micron) will be able to retain the scent liquid within the scent collecting chamber under normal air pressure without accuratation by the bottom piezoelectric vibration plate.

19. The scent-bearing disk or cartridge of claim 7 wherein the scent collecting chamber further comprises an absorbent material for momentarily retaining scent to be released from the scent-bearing disk or cartridge.

20. The scent-bearing disk or cartridge of claim 14 wherein the electrostatic scent release apparatus further comprises:
corona discharge pin for creating corona discharge to ionize scent; and
wiring for connecting the corona discharge pin to an external voltage source.

21. The scent-bearing disk or cartridge of claim 15 wherein the ultrasonic nebulizing scent release apparatus further comprises:
piezoelectric plate for creating ultrasonic vibration to vaporize scent; and
wiring for connecting the piezoelectric plate to an external power source.

22. The scent-bearing disk or cartridge of claim 16 wherein the thermal scent release apparatus further comprises:
thermal heater plate for creating heat to vaporize scent; and
wiring for connecting the thermal heater plate to an external power source.

23. The scent-bearing disk or cartridge of claim 20 wherein the electrostatic scent release apparatus further comprises:
a grid opposite from the corona discharge pin for attracting ionized scent molecules to assist in the scent release process.

24. The scent-bearing disk or cartridge of claim 20 wherein the electrostatic scent release apparatus further comprises:
unipolar or bipolar high voltage source for creating corona discharge to ionize scent.

25. The scent-bearing disk or cartridge of claim 21 wherein the ultrasonic nebulizing scent release apparatus further comprises:
a corona discharge pin mounted above the piezoelectric plate for ionizing ultrasonic-vibrated scent molecules to assist in the scent release process.

26. The scent-bearing disk or cartridge of claim 22 wherein the thermal scent release apparatus further comprises:
a corona discharge pin mounted above the thermal heater plate for ionizing thermal-vaporized scent molecules to assist in the scent release process.

27. A method for releasing scent from a scent storage and release unit, wherein the electrostatic scent release unit comprises a scent storage region; a scent release unit; a scent conducting tube connecting the scent release unit to the scent storage region; an electrostatic scent release apparatus located in the scent release unit; and a scent release micro-pumping unit, the method comprising the following steps:
opening the scent storage region to permit scent to flow from the scent storage region to the scent release unit;
activating the scent release micro-pumping units to improve scent flow from the scent storage region to the scent collecting chamber;
energizing the electrostatic scent release apparatus, thereby ionizing the scent molecules in the scent collecting chamber; and
energizing an oppositely-charged grid to attract the ionized scent molecules and thereby draw them out of the scent release unit;
creating an air flow near the scent release unit to assist in the venting of the released scent to a user, thereby entraining the scent molecules in the air flow; and
venting the scent molecules entrained in the air flow to the user without making any scent deposit on the housing or surrounding.

28. The method of claim 27 comprising the following further step:
releasing a scent-neutralizing substance to neutralize the previously-released scent.

29. The method of claim 28 wherein the scent-neutralizing substance comprises ozone.

30. The method of claim 27 comprising the following further step:
releasing another scent after the previously-released scent has been neutralized by the scent-neutralizing substance.

31. The method of claim 30 comprising the following further step:
releasing another scent after the previously-released scent has been neutralized by the ozone.

32. The method of claim 27 comprising the following further step:
releasing another ion and ozone after the previously-released ionized scent has been entrained in the air flow; and
ionizing or degrading the scent retention over the pores of the nozzle plate, thereby removing any retention or residual of previously-released scent.

33. A method for releasing scent from a scent storage and release unit, wherein the ultrasonic nebulizing scent release unit comprises a scent storage region; a scent release unit; a scent conducting tube connecting the scent release unit to the scent storage region; an ultrasonic nebulizing scent release apparatus located in the scent release unit; and a scent release micro-pumping unit, the method comprising the following steps:
opening the scent storage region to permit scent to flow from the scent storage region to the scent release unit;
activating the scent release micro-pumping units to improve scent flow from the scent storage region to the scent collecting chamber;
energizing the ultrasonic nebulizing scent release apparatus, thereby vaporizing the scent molecules in the scent collecting chamber; and
creating an air flow near the scent release unit to assist in the venting of the released scent to a user, thereby entraining the scent molecules in the air flow; and
venting the scent molecules entrained in the air flow to the user without making any scent deposit on the housing or surrounding.

34. The method of claim 33 comprising the following further step:
energizing the ultrasonic nebulizing scent release apparatus, thereby vibrating the scent deposited within the special constructed cup or container indirectly through water or other non-erosive liquid;
creating an air flow near the scent release unit to assist in the venting of the released scent to a user, thereby entraining the vaporized scent in the air flow; and
venting the vaporized scent entrained in the air flow to the user.

35. A method for releasing scent from a scent storage and release unit, wherein the thermal scent release unit comprises a scent storage region; a scent release unit; a scent conducting tube connecting the scent release unit to the scent storage region; an thermal scent release apparatus located in the scent release unit; and a scent release micro-pumping unit, the method comprising the following steps:
opening the scent storage region to permit scent to flow from the scent storage region to the scent release unit;
activating the scent release micro-pumping units to improve scent flow from the scent storage region to the scent collecting chamber;
energizing the thermal scent release apparatus, thereby vaporizing the scent molecules in the scent collecting chamber; and
creating an air flow near the scent release unit to assist in the venting of the released scent to a user, thereby entraining the scent molecules in the air flow; and
venting the scent molecules entrained in the air flow to the user without making any scent deposit on the housing or surrounding.

36. The method of claim 27 comprising the following further step:
mixing multiple scent substances at the scent collecting chamber;
creating an air flow near the scent release unit to assist in the venting of the released scent to a user, thereby entraining the scent molecules in the air flow; and
venting the scent molecules entrained in the air flow to the user.

37. The method of claim 36 wherein the multiple scent substances comprises liquid, gel, or solid form.

38. The method of claim 27 comprising the following further step:
mixing multiple gaseous scent substances within the scent releasing unit;
creating an air flow near the scent release unit to assist in the venting of the released scent to a user, thereby entraining the scent molecules in the air flow; and
venting the scent molecules entrained in the air flow to the user.

39. The scent-bearing disk or cartridge of claim 1 wherein the scent storage region stores a plurality of therapeutic agents.

40. The scent-bearing disk or cartridge of claim 14 wherein the electrostatic scent release apparatus further comprises:
corona discharge pin for creating corona discharge to ionize therapeutic agent; and
increasing the level of electrostatic charge on the particle of the therapeutic agent to assist in depositing the inhalabe therapeutic agent within the respiratory tract of the user.

41. The method of claims 20, 21, and 22 wherein the external power source further comprises an electromagnetic wave inducible voltage or power generator.

42. A method for generating high voltage or power by a electromagnetic wave, the electromagnetic wave inducible voltage or power generator of claim 41 further comprising a receiver for receiving electromagnetic wave from a remote source, a inducible electrical currents generator, and a high voltage generating circuit transforming a low voltage into a high voltage for assisting in the scent release from electrostatic scent release apparatus, the method comprising the following steps:
receiving the electromagnetic wave by the receiver from a remote source;
inducing electrical currents within the generator;
activating the high voltage generating circuit for transforming a low voltage into a high voltage;
energizing the electrostatic scent release apparatus, thereby ionizing the scent molecules;
creating an air flow near the scent release unit to assist in the venting of the released scent to a user, thereby entraining the scent molecules in the air flow; and
venting the scent molecules entrained in the air flow to the user without making any scent deposit on the housing or surrounding.

43. The method of claim 42 wherein the electromagnetic wave scent release apparatus further comprises:
creating high energy electromagnetic wave to ionize the scent molecules in the scent collecting chamber.

44. The method of claim 27 comprising the following further step:
releasing controlled amount of ozone in a controlled frequency after previously-released scent;
creating an air flow faster than the previously-released scent flow to assist in the process of catching up the previously-released scent within a controlled range, thereby neutralizing or degrading the previously-released scent molecules in the air flow; and
preventing the scent molecules from trespassing over the controlled range adjusted by the user.

45. The method of claim 44 comprising the following further step:
rotating automatically scent release unit in various angle;
releasing controlled amount of ozone in a controlled frequency to assist in neutralizing or degrading of the previously-released scent, dust, or other particles within the controlled range.

46. A method for using a scent-bearing and multimedia disk or cartridge having scent release means, wherein the method uses a separate scent recovery/release and multimedia playback system to release scent and recover multimedia and scent release control information from the scent-bearing and multimedia cartridge, and wherein the method comprises the following steps:
receiving an input command from a user to initiate scent release and multimedia playback;
recovering digital scent release and multimedia playback control information corresponding to the user input command from the scent-bearing and multimedia portion of the disk;
initiating multimedia recovery and playback corresponding to the multimedia segment selected by the user;
interpreting the identity, time, and duration of scent release reflected in the digital scent release control information; and
releasing the proper scents at the proper time and for the proper duration from the scent-bearing and multimedia cartridge using scent release means.

47. The method of claim 46 wherein a plurality of scents are released simultaneously.

48. The method of claim 46 comprising the additional following step:
releasing a scent neutralizing agent following the release of a scent.

49. The method of claim 46 comprising the following further step:
releasing a controlled amount of energizing or refreshing scent at a user-inputted preprogram time of the scent recovery/release system to assist in increasing alertness of the user.

50. The scent-bearing cartridge of claim 1 wherein the scent recovery/release system further comprises:
a security system integrated scent recovery/release system for releasing scent to assist in the alarm or alert operation for the user.

51. The scent-bearing cartridge of claim 1 wherein the scent recovery/release system further comprises:
a driver fatigue monitoring system integrated scent recovery/release system for releasing scent to assist in increasing the alertness or preventing drowsiness of the driver while the monitoring system has been triggered.

52. The scent-bearing cartridge of claim 1 wherein the scent recovery/release system further comprises:
a driver handlebar integrated control unit for sending control signal to the scent recovery/release system for releasing scent in the car while a manual input is from a driver.

53. The scent-bearing cartridge of claim 1 wherein the scent recovery/release system further comprises:
a driver seatbelt integrated fatigue monitoring system for sending control signal to the scent recovery/release system for releasing scent to the driver while the driver's fatigue condition has been detected by the monitor.

54. The scent recovery/release system of claim 53 wherein the driver seatbelt integrated fatigue monitoring system further comprises:
a heartbeat, pulse, breath, and blood pressure monitoring system to assist in monitoring the drossiness of the driver for sending control signal to the scent recovery/release system for releasing scent to the driver while the driver's fatigue condition has been detected by the monitor.

55. The scent-bearing cartridge of claim 1 wherein the scent recovery/release system further comprises:
a sensor integrated scent recovery/release system for releasing scent in response to an electromagnetic wave, a vibration, a sound, an infrared detection, or other means of determining activation within the vicinity of the device.

56. A scent-bearing and multimedia disk or cartridge comprising:
a housing containing an interior cavity, the housing having at least one opening for releasing scent;
at least one scent release unit, wherein the scent release unit comprises
a scent storage unit for storing scent, the scent storage unit enclosed on three sides, the scent storage unit having an upwardly facing opening for releasing scent;
an absorbent material for retaining scent before scent release operations; and
an electrostatic, a ultrasonic nebulizing, or a thermal scent release apparatus for ionizing or vaporizing scent to be released from the scent storage unit.

57. A method for releasing scent from a scent-bearing and multimedia disk or cartridge, the scent-bearing and multimedia disk or cartridge storing scent in a scent storage unit, the scent storage unit further comprising a cover for covering the scent release unit when scent is not being released from the scent-bearing and multimedia disk or cartridge and electrostatic scent release apparatus for assisting in the scent release, the method comprising the following steps:
opening the cover to permit scent release to a region immediately adjacent to the scent release unit;
energizing the electrostatic scent release apparatus, thereby ionizing the scent molecules;
energizing an oppositely-charged grid to attract the ionized scent molecules and thereby draw them out of the scent storage unit;
creating an air flow near the scent storage unit to assist in the venting of the released scent to a user, thereby entraining the scent molecules in the air flow; and
venting the scent molecules entrained in the air flow to the user.

58. A method for releasing scent from a scent-bearing and multimedia disk or cartridge, the scent-bearing and multimedia disk or cartridge storing scent in a scent storage unit, the scent storage unit further comprising a cover for covering the scent release unit when scent is not being released from the scent-bearing and multimedia disk or cartridge and ultrasonic nebulizing scent release apparatus for assisting in the scent release, the method comprising the following steps:
opening the cover to permit scent release to a region immediately adjacent to the scent release unit;
energizing the ultrasonic nebulizing scent release apparatus, thereby vaporizing the scent molecules;
creating an air flow near the scent storage unit to assist in the venting of the released scent to a user, thereby entraining the scent molecules in the air flow; and
venting the scent molecules entrained in the air flow to the user.

59. A method for releasing scent from a scent-bearing and multimedia disk or cartridge, the scent-bearing and multimedia disk or cartridge storing scent in a scent storage unit, the scent storage unit further comprising a cover for covering the scent release unit when scent is not being released from the scent-bearing and multimedia disk or cartridge and thermal scent release apparatus for assisting in the scent release, the method comprising the following steps:
opening the cover to permit scent release to a region immediately adjacent to the scent release unit;
energizing the thermal scent release apparatus, thereby vaporizing the scent molecules;
creating an air flow near the scent storage unit to assist in the venting of the released scent to a user, thereby entraining the scent molecules in the air flow; and
venting the scent molecules entrained in the air flow to the user.

60. The scent-bearing and multimedia disk or cartridge of claim 56 wherein the scent release means comprises a single release valve.

61. A scent and multimedia-bearing cartridge for use with a separate multimedia playback and scent recovery system, the scent and multimedia-bearing cartridge comprising:
a scent storage region for storing at least one scent;
a scent release unit;
a scent conducting tube connecting the scent storage region to the scent release unit, wherein during scent release operations scent flows from the scent storage region to the scent release unit; and
an encapsulated multimedia storage medium for storing multimedia and scent release control information.

62. The scent and multimedia-bearing cartridge of claim 61 wherein the multimedia storage structure comprises a Type II DVD-RAM-like cassette device, the Type II DVD-RAM-like cassette having a housing structure encapsulating a removable DVD-RAM or DVD disc.

63. The scent and multimedia -bearing cartridge of claim 61 wherein the scent storage region stores a plurality of scents.

64. A micro-pump and electrostatic scent release nosepiece for releasing scent directly to the nose and/or mouth of a user, the micro-pump and electrostatic scent release nosepiece further comprising:
a nose-bridge-mounted control unit placed directly above the nose-bridge of the user for transmitting the control signal;
flexible-plastic-covered wirings to connect the scent storage and micro-pump release unit;
a scent storage and micro-pump release unit, the scent storage and micro-pump release unit further comprising:
a nosepiece housing that comfortably fits over the nose of a user;
at least one scent storage unit;
at least one micro-pump unit; and
at least one electrostatic scent release unit

65. The micro-pump and electrostatic scent release nosepiece of claim 64, wherein the at least one scent storage unit, at least one micro-pump unit and at least one electrostatic scent release unit are positioned adjacent to the nose to release scent directly to the nose of the user.

66. The micro-pump and electrostatic scent release nosepiece of claim 64, wherein the micro-pump means further comprises:
a micro-pump unit for releasing scent from the at least one scent storage unit;
a scent conducting tube connecting to at least one scent storage unit to the micro-pump unit; and
wirings connecting the micro-pump unit and the control unit.

67. The micro-pump and electrostatic scent release nosepiece of claim 64, wherein the electrostatic scent release means further comprises:
an electrostatic discharge needle for ionizing scent to be released from the at least one scent storage means;
a source of electric potential to supply electricity to the electrostatic discharge needle; and
wiring connecting the electrostatic discharge needle and the source of electric potential.

68. The micro-pump and electrostatic scent release nosepiece of claim 64 wherein the scent storage unit stores a plurality of therapeutic agents.

69. A multimedia playback and electrostatic scent release desktop diffuser for releasing scent directly to the user, the multimedia playback and electrostatic scent release desktop diffuser further comprising:
a display screen mounted on the housing to display multimedia information;
a housing for containing scent-bearing cartridge;
speakers to play back auditory information;
a user input pad and interface; and
at least one electrostatic scent release unit.

70. The multimedia playback and electrostatic scent release desktop diffuser of claim 69, wherein the scent release means further comprises:
a special design nozzle head for pumping scent through the activation of a solenoid during scent releasing operation to improve the flow of scent from the scent storage region to the scent release unit.

71. The multimedia playback and electrostatic scent release desktop diffuser of claim 69, wherein scent-bearing disk or cartridge means further comprises:
an independently replaceable and customizing scent storage region for refilling and rearranging at least one scent by the user.

72. The method of claim 71 wherein during customizing the scent storage region means the scents can be related to each other in a manner which is not dependent on some other media, and/or are not designed to be used simultaneously with other media.

73. A multimedia playback and electrostatic scent release desktop diffuser for releasing scent directly to the user, the multimedia playback and electrostatic scent release desktop diffuser further comprising:
a display screen mounted on the housing to display multimedia information;
a separate multimedia playback and scent recovery system;
a housing for containing scent-bearing cartridge;
speakers to play back auditory information;
a user input means; and
at least one electrostatic scent release unit.

74. The multimedia playback and electrostatic scent release desktop diffuser of claim 73, wherein the separate multimedia playback and scent recovery system further comprises:
a scent related content and recovery information for the scent-bearing disk or cartridge is provided by a separated multimedia storage medium from the multimedia playback system.

75. The multimedia playback and electrostatic scent release desktop diffuser of claim 74, wherein the separate multimedia playback and scent recovery system further comprises:
a scent related content and scent release control information for the scent-bearing disk or cartridge is provided by a remote source(e.g. internet, server, LAN, or wireless LAN).

76. The multimedia playback and electrostatic scent release desktop diffuser of claim 73, wherein the scent-bearing disk or cartridge further comprises:
a graphical or text labeling on the storage case to provide user with the scent related content, texts or recovery information.

77. The multimedia playback and electrostatic scent release desktop diffuser of claim 73, wherein the user input means further comprises:
inputting the graphical or text labeling information into the diffuser by the user.

78. The method of claim 77 wherein during the user inputting of scent related content and texts information the scents can be related to each other in a manner which is not dependent on some other media, and/or are not designed to be used simultaneously with other media.

79. A multimedia playback and electrostatic scent release desktop diffuser for releasing scent directly to the user, the multimedia playback and electrostatic scent release desktop diffuser further comprising:
a docking structure for holding a hand-held device;
a communication and power supply port to connect between the multimedia playback and electrostatic scent release desktop diffuser and the hand-held device;
a housing for containing scent-bearing cartridge;
speakers to play back auditory information;
a user input pad and interface; and
at least one electrostatic scent release unit.

80. The multimedia playback and electrostatic scent release desktop diffuser of claim 79, wherein the hand-held device further comprises:
a receiver for receiving wireless signal from a remote source; and
a transceiver to transmit the control signal to the multimedia playback and scent recovery system through the communication and power supply port.

81. A multimedia playback and electrostatic scent release personal diffuser for releasing scent directly to the user, the multimedia playback and electrostatic scent release personal diffuser further comprising:
an attachment means placed directly adjacent to the housing of the diffuser;
a display screen mounted on the housing to display multimedia information;
an earphone port to output auditory information;
a housing for containing scent-bearing cartridge;
a user input pad and interface; and
at least one electrostatic scent release unit.

82. The multimedia playback and electrostatic scent release personal diffuser of claim 81, wherein the attachment means further comprises a clip structure, a drawstring, a interlocking, a loop of fabric, a VELCRO- like fabric patches, or a mechanical snap.

83. The multimedia playback and electrostatic scent release personal diffuser of claim 81, wherein the electrostatic scent release means further comprises:
an electrostatic discharge needle for ionizing scent to be released from the at least one scent storage unit;
a source of electric potential to supply electricity to the electrostatic discharge needle; and
wiring connecting the electrostatic discharge needle and the source of electric potential.

84. The multimedia playback and electrostatic scent release personal diffuser of claim 81, wherein the electrostatic scent release means further comprises:
a fan and ducting to create an airflow in the vicinity of the electrostatic discharge needle to assist in entraining scent ionized by the electrostatic discharge needle.

85. The multimedia playback and electrostatic scent release personal diffuser of claim 81 wherein the scent storage unit stores a plurality of therapeutic agents.
